(19) United States
(12) Patent Application Publication
(10) Pub. No.: US 2021/0270848 A1
(43) Pub. Date: Sep. 2, 2021

(54) METHOD FOR DIAGNOSING DEMENTIA OR DETERMINING THE RISK OF DEVELOPING DEMENTIA

(71) Applicant: UNIVERSITY OF LEICESTER, Leicester (GB)

(72) Inventors: Elizabeta MUKAETOVA-LADINSKA, Leicester (GB); Mike CATT, Newcastle (GB)

(21) Appl. No.: 17/274,399

(22) PCT Filed: Sep. 6, 2019
(86) PCT No.: PCT/GB2019/052487

§ 371 Date: Mar. 8, 2021

(30) Foreign Application Priority Data
Sep. 12, 2018 (GB) 1814807.2

(51) Int. Cl. G01N 33/68; A61K 31/445; A61K 31/27; A61K 31/55; A61K 31/473; A61K 31/13

(52) U.S. Cl. CPC G01N 33/6896; A61K 31/445; A61K 31/27; A61K 31/55; A61K 31/473; G01N 2333/775; G01N 2800/2814; G01N 2800/52; G01N 2800/50; G01N 2496/00; G01N 2333/4709; A61K 31/13

(57) ABSTRACT

The present invention provides novel biomarkers for dementia. Methods for diagnosing dementia or the risk of developing dementia, or for monitoring dementia progression are also provided. The invention also provides methods for determining the therapeutic effect of appropriate treatment regimens or determining a subjects compliance or adherence with a prescribed treatment regimen. A method for monitoring changes in cognition in a subject having or suspected of having dementia is also provided. Corresponding kits, assay devices and uses are also provided.

The present invention provides novel biomarkers for dementia. Methods for diagnosing dementia or the risk of developing dementia, or for monitoring dementia progression are also provided. The invention also provides methods for determining the therapeutic effect of appropriate treatment regimens or determining a subject's compliance or adherence with a prescribed treatment regimen. A method for monitoring changes in cognition in a subject having or suspected of having dementia is also provided. Corresponding kits, assay devices and uses are also provided.

### BACKGROUND

An Overview of Dementia

Dementia is an ageing disorder affecting 5% of older people (>65 years) and up to 40% of those aged 80 years or more (Luzny et al., 2014). An increase in the prevalence of dementia has been witnessed worldwide due to the rapidly ageing population. Currently, it is estimated that there are 35.6 million people living with dementia around the world, and this is predicted to increase to 115 million by 2050 (Bunn et al., 2014).

The most common type of dementia is Alzheimer's disease (AD) which affects approximately 60% of dementia patients in Western countries and 6% of people over the age of 65 (Rizzi et al., 2014 and Wurtman, 2015). Alzheimer's disease is not yet fully understood, but clinically it is characterised by memory loss, cognitive decline and behavioural problems (Chiam et al., 2015). Other common forms of dementia include Vascular dementia (VaD), mixed dementia and Dementia with Lewy Bodies (DLB) (Rizzi et al., 2014).

The neuropathological changes observed in different forms of dementia are typically attributable to an altered protein which causes damage to surrounding neurons. Pathologically, AD is caused by the deposition of proteins around and inside the neurons which eventually leads to cell death and neuronal loss throughout the brain tissue (Kumar et al., 2014). The two proteins involved in AD are beta-amyloid (Aβ) and hyper-phosphorylated and truncated tau, which form extracellular plaques and intracellular neurofibrillary tangles (Alzheimer's Association, 2011). However, the specific mechanism leading to the build-up of plaques and tangles is not yet known (Rembach et al., 2014). In Vascular dementia, the neuropathological changes associated with cognitive impairment are not yet clearly defined, and loss of synaptic and tau proteins has been recently linked, in addition to the underlying vascular and ischaemic changes leading to neuronal cell death in the hippocampus and temporal lobe of affected individuals (Foster et al., 2014, Mukaetova-Ladinska et al, 2015).

In contrast, the neuropathology of Lewy body diseases (including DLB and Parkinson's disease dementia) is characterised by widespread distribution of intracellular Lewy bodies, consisting of aggregated α-synuclein, a heat stable synaptic protein (Kim et al., 2014).

Diagnosis of Dementia

Presently, a definite diagnosis of dementia can only be established post-mortem, based on identifying histochemical changes and protein dispositions characteristic for distinct dementia subtypes within the brain (Khan et al., 2015). However, in the clinical setting, the behavioural changes and cognitive dysfunction of a dementia sufferer provide evidence for a probable and possible diagnosis of dementia (Table 1).

TABLE 1
A summary of various dementia subtypes and the symptoms which
may be seen for each subtype (Hall and Finger 2015, Boot 2015 and
Mukaetova-Ladinska 2015).

| Dementia type | Clinical characteristics |
|---|---|
| Alzheimer's | Gradual memory loss, is often the earliest symptom. |
| Disease | Mood & personality changes, and paranoia. |
| | Disorientation and misinterpretation of spatial |
| | relationships. Higher cognitive functions, e.g. |
| | reading, writing, language command etc. are |
| | impaired at later stages of dementia. |
| Vascular | Step wise memory loss, mood changes and apathy, |
| Dementia | impairment inlanguage and information processing, |
| | decision making and visuospatial deficits. Symptoms |
| | may occur rapidly after stroke or damage to small |
| | vessels. Severity depends on location and extent. |
| DLB | Fluctuations in cognitive function resembling |
| | delirium. Visual hallucinations and illusions, as |
| | well as sleep disturbances [e.g. Rapid Eye |
| | Movement (REM) sleep problems] anxiety and |
| | depression. Symptoms of Parkinsonism where tremor, |
| | bradykinesia, poor gait and postural instabilities |
| | appear after onset of memory problems. |
| Parkinson's | Parkinsonism including akynesia, bradykinesia, |
| Disease | tremor, poor gaitand postural instabilities which |
| Dementia | occur before symptoms affecting cognitive function. |
| | Similar symptoms to DLB. |
| Frontotemporal | Affects predominantly younger people (<50-60 years |
| Dementia | old). Characteristic changes in behaviour and |
| | personality, with disinhibition and loss of restraint in |
| | personal relations and sociallife. Memory normally |
| | affected in later stages. Psychosis, hallucinations and |
| | delusions seen in 20% of cases. |

Dementia pathology commences approximately a decade before the overt clinical symptoms of dementia arise. Many symptoms may mirror those which accompany natural ageing in the initial stages of cognitive impairment (Maki and Yamaguchi, 2014). For these reasons, diagnosing dementia can often be a long and arduous process, and it has been stated that almost two thirds of dementia patients are not diagnosed (Chiam et al., 2015). Further complications emerge due to the heterogenic expression of clinical symptoms, within the same subtypes of dementia, which can result in misdiagnosis (Masellis et al., 2013). Once diagnosed, it is critical for patient treatment that the specific sub-type of dementia is identified (Table 1).

At present, diagnosis begins with recognising symptoms in an attempt to exclude other treatable, reversible causes of dementia, such as hypothyroidism, infection, anaemia, brain injury, nutritional deficiency, pharmacological causes, metabolic and/or hormonal disorders (Gupta et al., 2012). This is also supported by detailed clinical information obtained from both the patient and their next of kin or carers. The patient is then monitored through primary care (medical examinations, e.g. physical and neurological assessments, and additional evaluations such as chest radiography, electrocardiography, and laboratory tests) to determine the severity of the symptoms (Cooper and Greene, 2005). These include routine blood tests to assess homocysteine, folic acid and thyroid stimulating hormone levels, and urinalysis (Tsolaki, 2014). Subsequent referral to specialist memory services focuses on defining the dementia sub-type in order to construct a treatment plan, e.g. anti-dementia drugs (such as cholinesterase inhibitors, NMDA-agonists) and other interventions, including neuroleptic and antidepressant treatments for behavioural and mood changes (Lunzy et al., 2014). Neuroimaging, including Computerised Tomography (CT), Magnetic Resonance Imaging (MRI) and Single Photon Emission Computer Tomography (SPECT) brain scans, is widely used to aid the dementia differential diagnosis.

Currently there are only a small number of recognised biomarkers found in cerebrospinal fluid (CSF) which are generally accepted and used in combination with other tests, such as neuroimaging, for the diagnosis of dementia. The recommended CSF biomarkers are largely orientated around the proteins involved in the pathology of AD. These include Aβ 1-42 (Aβ), total tau (t-tau) and phosphorylated tau (p-tau) concentrations. Table 2 summarises the types of biomarkers found in the CSF and how their concentrations are affected in dementia.

TABLE 2
Summarising established biomarkers in CSF and the change in
concentration observed in cases of dementia (De Sole etal., 2013).

| Biomarker | Levels in Dementia |
|---|---|
| t-Tau | Up |
| Aβ | Down |
| p-Tau | Up |
| BACE-1 | Up |
| sAPP1/sAPP2 | Up |
| TKL-40 | Up |
| AβOligomers | Up |

CSF Aβ is inversely correlated with plaque progression, whereas t-tau mirrors the amount of neuronal damage. p-Tau reflects the number of neurofibrillary tangles consisting of hyper-phosphorylated tau protein that causes cytoskeletal instability and tau protein aggregation into paired helical filaments (PHFs) (Skillback et al., 2015). These changes can be observed in the CSF as early as pre-clinical mild cognitive impairment (MCI) and are greatly enhanced once disease has progressed to the overt dementia stages (Sweeney et al., 2015). CSF t-tau and p-tau alone have high sensitivity (80-93%) and specificity (82%-90%) for AD (Skillback et al., 2015).

Although CSF biomarkers may provide useful information about the biochemical changes that occur in the brain tissue of dementia sufferers, their use in routine clinical practice has a number of drawbacks. The invasive procedure via which CSF is obtained (e.g. lumbar puncture) is deemed unethical in many countries. Another obstacle is that CSF cannot be collected in large quantities, which limits its usage (Chiam et al., 2015). CSF biochemical analysis is also costly and cannot be used globally. In addition, there is heterogeneity in the results obtained from different laboratories, and the results are not reproducible. Overall, these disadvantages prevent CSF biomarkers from reaching status as ideal biomarkers for dementia.

Blood biomarkers have clinical importance in diagnosing a wide range of diseases including diabetes, cancer and immune disorders. The research into potential blood biomarkers for dementia has focussed on plasma and serum, but as of yet there has been no breakthrough and results from different laboratories provide no consensus (Snyder et al., 2014). There are several reasons behind this, including the limited ability of brain proteins to penetrate the peripheral circulation due to the blood-brain barrier (BBB) (Michell et al., 2005). The BBB separates the brain and blood, with the purpose of protecting neurons and other brain cellular components from toxic proteins, cells, pathogens and metals. It possesses specialised transport systems which carry nutrients across the barrier via specific binding sites. This crucial system can prevent proteins from crossing the BBB (Sweeney et al., 2015), causing the concentration of originating brain proteins in the blood to be lower than that of the CSF. Further, any proteins that are successful in penetrating the barrier are susceptible to degradation on entering the blood (Michell et al., 2005).

There is a need to identify new dementia biomarkers that are reliable, reproducible and able to differentiate between dementia subtypes.

### BRIEF SUMMARY OF THE DISCLOSURE

The inventors have surprisingly found a number of biomarkers for dementia in whole blood. Advantageously, whole blood biomarkers are easily obtainable and are available in large quantities for precise analysis and clinical application and therefore may be used for early and rapid diagnosis of dementia. The invention therefore provides a new means for improving dementia diagnosis and monitoring dementia progression. In addition, it provides a new opportunity for treatment to commence at much earlier stages of dementia disease progression.

In one aspect, the invention provides an in vitro method for diagnosing dementia or determining the risk of developing dementia in a subject, the method comprising the steps of:

a) providing a whole blood sample from the subject;

b) determining the level of one or more biomarker in the whole blood sample, wherein the one or more biomarker is selected from the group consisting of clusterin, alpha synuclein (AS) and amyloid precursor protein (APP);

c) comparing the level of the one or more biomarker with the level of the same biomarker in a control sample or with a pre-determined reference level for the same biomarker to identify an increase or decrease in a level of the one or more biomarker in the sample of the subject compared to the control sample or pre-determined reference value;

d) identifying a subject as having dementia or as having an increased risk of developing dementia if the comparison in step c) indicates that the subject has one or more of the following: a change in the level of clusterin compared to the control sample or the pre-determined reference level; an increased level of alpha-synuclein compared to the control sample or the pre-determined reference level; or a decreased level of amyloid precursor protein compared to the control sample or the pre-determined reference level.

Suitably, step b) comprises the determining the level of at least two biomarkers selected from the group consisting of clusterin, alpha-synuclein and amyloid precursor protein.

Suitably, the cause of dementia is a dementia-related neurological disorder, such as Alzheimer's disease (AD), Vascular dementia (VaD) or Dementia with Lewy Bodies.

Suitably, the method comprises identifying a subject with a decreased level of clusterin compared to the control sample or the pre-determined reference level as having Alzheimer's disease or as having increased risk of developing Alzheimer's disease.

Suitably, the method comprises identifying a subject with an increased level of clusterin compared to the control sample or the pre-determined reference level as having Vascular dementia or as having increased risk of developing Vascular dementia.

Suitably, the level of biomarker is determined at the protein level, optionally using a process selected from the group consisting of immunoblotting, lateral flow assay, ELISA assay, protein microarray and mass spectrometry.

Suitably, the control sample is obtained from a non-demented control subject.

Suitably, the pre-determined reference level is the average level of the biomarker in a non-demented control subject.

Suitably, the subject is a human.

Suitably, the method further comprises selecting a treatment for the subject based on the comparison of the level of the biomarker with the control sample or with the pre-determined reference level.

Suitably, the method further comprises administering the selected treatment to the subject, optionally wherein the selected treatment comprises an effective amount of at least one anti-dementia compound.

Suitably, the anti-dementia compound is:

a) a cholinesterase inhibitor, optionally wherein the cholinesterase inhibitor is selected from the group consisting of donepezil, rivastigmine, galantamine, tacrine, or salts thereof, and/or

b) an NMDA antagonist, optionally wherein the NMDA antagonist is memantine.

In another aspect, the invention provides a kit for diagnosing dementia or determining the risk of developing dementia in a subject, comprising:

(i) a detectably labelled agent that specifically binds to clusterin; and

(ii) one or more of:
a) a detectably labelled agent that specifically binds to alpha-synuclein; and
b) a detectably labelled agent that specifically binds to amyloid precursor protein (APP).

Suitably, the kit comprises a) and b).

Suitably, the kit comprises one or more reagents for detecting the detectably labelled agent(s).

In another aspect, the invention provides an assay device for diagnosing dementia or determining the risk of developing dementia in a subject, the device comprising a surface with at least two detectably labelled agents located thereon, wherein the at least two detectably labelled agents are:

(i) a detectably labelled agent that specifically binds to clusterin; and

(ii) one or more of:
a) a detectably labelled agent that specifically binds to alpha-synuclein; and
b) a detectably labelled agent that specifically binds to amyloid precursor protein (APP).

Suitably, the device comprises a) and b).

Suitably, the at least two detectably labeled agents are located in separate zones on the surface.

In another aspect, the invention provides the use of one or more biomarkers selected from the group consisting of clusterin, alpha-synuclein and amyloid precursor protein (APP) as a whole blood biomarker for dementia.

Suitably, the cause of the dementia is a dementia-related neurological disorder, such as Alzheimer's disease (AD), Vascular dementia (VaD) or Dementia with Lewy Bodies (DLB).

In another aspect, the invention provides an in vitro method for monitoring dementia progression in a subject, the method comprising the steps of:

i) determining the level of one or more biomarker in a whole blood sample from the subject in accordance with method steps a) to c) of any one of the methods described above; and

ii) repeating step i) for the same subject after a time interval;

iii) comparing the biomarker levels identified in i) with the biomarker levels identified in ii), wherein a change in the biomarker levels from i) to ii) is indicative of a change in dementia status/progression in the subject.

In another aspect, the invention provides an in vitro method for determining the therapeutic effect of a treatment regimen for dementia, the method comprising:

a) providing a whole blood sample from the subject;

b) determining the level of one or more biomarker in the whole blood sample, wherein the one or more biomarker is selected from the group consisting of clusterin, alpha-synuclein and amyloid precursor protein (APP);

c) repeating steps a) and b) using a whole blood sample obtained from the subject after treatment for a time interval; and

d) comparing the level of biomarker determined in step b) to that determined in step c), and identifying that the treatment regimen has a therapeutic effect if one or more of the following is observed: there is a change in the level of clusterin after treatment; there is an increase in the level of alpha-synuclein after treatment; or there is an increase in the level of amyloid precursor protein after treatment.

Suitably, the cause of the dementia is a dementia-related neurological disorder such as Alzheimer's disease (AD) or Vascular dementia (VaD).

Suitably, step d) comprises identifying that the treatment regimen has a therapeutic effect if the level of clusterin in c) compared to b) is increased and the subject has Alzheimer's disease or is at increased risk of developing Alzheimer's disease.

Suitably, step d) comprises identifying that the treatment regimen has a therapeutic effect if the level of clusterin in c) compared to b) is decreased and the subject has Vascular dementia or is at increased risk of developing Vascular dementia.

In another aspect, the invention provides an in vitro method for determining a subject's compliance or adherence with a prescribed treatment regimen for dementia, the method comprising:

a) providing a whole blood sample from the subject;

b) determining the level of one or more biomarker in the whole blood sample, wherein the one or more biomarker is selected from the group consisting of clusterin, alpha-synuclein and amyloid precursor protein (APP);

c) repeating steps a) and b) after a time interval using a whole blood sample obtained from the subject after the prescribed start of treatment regimen; and

d) comparing the level of biomarker determined in step b) to that determined in step c), and identifying that the subject has complied or adhered with the prescribed treatment regimen if one or more of the following is observed: there is a change in the level of clusterin after treatment with the medicament; there is an increase in the level of alpha-synuclein after treatment; or there is an increase in the level of amyloid precursor protein after treatment.

Suitably, the cause of the dementia is a dementia-related neurological disorder such as Alzheimer's disease (AD) or Vascular dementia (VaD).

Suitably, step d) comprises identifying that the subject has complied or adhered with the prescribed treatment regimen if the level of clusterin in c) compared to b) is increased and the subject has Alzheimer's disease or is at increased risk of developing Alzheimer's disease.

Suitably, step d) comprises identifying that the subject has complied or adhered with the prescribed treatment regimen if the level of clusterin in c) compared to b) is decreased and the subject has Vascular dementia or is at increased risk of developing Vascular dementia.

Suitably, the treatment regimen comprises at least one anti-dementia compound.

Suitably, the anti-dementia compound is:

a) a cholinesterase inhibitor, optionally wherein the cholinesterase inhibitor is selected from the group consisting of donepezil, rivastigmine, galantamine, tacrine, or salts thereof, and/or

b) an NMDA antagonist, optionally wherein the NMDA antagonist is memantine.

Suitably, the level of at least two biomarkers selected from the group consisting of clusterin, alpha-synuclein and amyloid precursor protein is determined and compared.

Suitably, the level of biomarker is determined at the protein level, optionally using a process selected from the group consisting of immunoblotting, lateral flow assay, ELISA assay, protein microarray and mass spectrometry.

Suitably, the subject is a human.

In another aspect, the invention provides the use of one or more biomarkers selected from the group consisting of clusterin, alpha-synuclein and amyloid precursor protein (APP) as a whole blood biomarker for assessing cognition in a subject having or at risk of having dementia.

Suitably, the cause of the dementia is a dementia-related neurological disorder such as Alzheimer's disease (AD), Vascular dementia (VaD) or Dementia with Lewy bodies (DLB).

In another aspect, the invention provides an in vitro method for monitoring changes in cognition in a subject having or at risk of having dementia, the method comprising the steps of:

i) performing the following steps:
a) providing a whole blood sample from the subject;
b) determining the level of one or more biomarker in the whole blood sample, wherein the one or more biomarker is selected from the group consisting of clusterin, alpha synuclein (AS) and amyloid precursor protein (APP);
c) comparing the level of the one or more biomarker with the level of the same biomarker in a control sample or with a pre-determined reference level for the same biomarker to identify an increase or decrease in a level of the one or more biomarker in the sample of the subject;

ii) repeating i) for the same subject after a time interval;

iii) comparing the biomarker levels identified in i) with the biomarker levels identified in ii), and identifying a reduction in cognitive score if the comparison in step iii) indicates that the subject has one or more of the following: a change in the level of clusterin over the time interval; an increase in the level of alpha-synuclein over the time interval; or a decrease in the level of amyloid precursor protein over the time interval.

Suitably, the level of at least two biomarkers selected from the group consisting of clusterin, alpha-synuclein and amyloid precursor protein are determined and compared over the time interval.

Suitably, the cause of the dementia is a dementia-related neurological disorder such as Alzheimer's disease (AD), Vascular dementia (VaD) or dementia with Lewy bodies.

Suitably, the level of biomarker is determined at the protein level, optionally using a process selected from the group consisting of immunoblotting, lateral flow assay, ELISA assay, protein microarray and mass spectrometry.

Suitably, the control sample is obtained from a non-demented control subject.

Suitably, the pre-determined reference level is the average level of the biomarker in a non-demented control subject.

Suitably, the subject is a human.

In another aspect, the invention provides a method of diagnosing and treating dementia in a subject, the method comprising the steps of:

a) providing a whole blood sample from the subject;

b) determining the level of one or more biomarker in the whole blood sample, wherein the one or more biomarker is selected from the group consisting of clusterin, alpha synuclein (AS) and amyloid precursor protein (APP);

c) comparing the level of the one or more biomarker with the level of the same biomarker in a control sample or with a pre-determined reference level for the same biomarker to identify an increase or decrease in a level of the one or more biomarker in the sample of the subject compared to the control sample or pre-determined reference value;

d) diagnosing the subject with dementia if the comparison in step c) indicates that the subject has one or more of the following: a change in the level of clusterin compared to the control sample or the pre-determined reference level; an increased level of alpha-synuclein compared to the control sample or the pre-determined reference level; or a decreased level of amyloid precursor protein compared to the control sample or the pre-determined reference level; and

e) administering an effective amount of at least one anti-dementia compound to the diagnosed subject.

Appropriate anti-dementia compounds are discussed elsewhere herein.

In another aspect, the invention provides a method of treating a subject with dementia, the method comprising administering an effective amount of at least one anti-dementia compound to the patient, wherein the patient has been diagnosed as having dementia using a method described elsewhere herein.

In another aspect, the invention provides a method of detecting dementia in a subject, the method comprising:

a. obtaining a whole blood sample from a human patient and

b. detecting the level of one or more biomarker in the whole blood sample, wherein the one or more biomarker is selected from the group consisting of clusterin, alpha synuclein (AS) and amyloid precursor protein (APP), by contacting the whole blood sample with an appropriate antibody specific to the biomarker of interest and detecting binding between the biomarker and the corresponding antibody (i.e. clusterin binding to an anti-clusterin antibody; alpha synuclein (AS) binding to an anti-AS antibody or amyloid precursor protein (APP) binding to an anti-APP antibody).

In another aspect, the invention provides a method of monitoring dementia progression in a subject and treating the subject, the method comprising the steps of:

i) determining the level of one or more biomarker in a whole blood sample from the subject in accordance with method steps a) to c) of any one of the methods described above; and

ii) repeating step i) for the same subject after a time interval;

iii) comparing the biomarker levels identified in i) with the biomarker levels identified in ii), wherein a change in the biomarker levels from i) to ii) is indicative of a change in dementia status/progression in the subject; and

iv) administering an effective amount of at least one anti-dementia compound to a subject identified as having dementia progression.

In another aspect, the invention provides a method of treating a subject with dementia, the method comprising administering an effective amount of at least one anti-dementia compound to the patient, wherein the patient has been identified as having dementia progression using a method described elsewhere herein.

In another aspect, the invention provides a method of monitoring dementia progression in a subject, the method comprising:

a. obtaining a whole blood sample from a human patient and

b. detecting the level of one or more biomarker in the whole blood sample, wherein the one or more biomarker is selected from the group consisting of clusterin, alpha synuclein (AS) and amyloid precursor protein (APP), by contacting the whole blood sample with an appropriate antibody specific to the biomarker of interest and detecting binding between the biomarker and the corresponding antibody (i.e. clusterin binding to an anti-clusterin antibody; alpha synuclein (AS) binding to an anti-AS antibody or amyloid precursor protein (APP) binding to an anti-APP antibody);

c. repeating steps a and b for the same subject after a time interval; and

d. comparing the biomarker levels identified in b) with the biomarker levels identified in c), wherein a change in the biomarker levels from b) to c) is indicative of a change in dementia status/progression in the subject; and

e. administering an effective amount of at least one anti-dementia compound to a subject identified as having dementia progression.

In another aspect, the invention provides a method for determining the therapeutic effect of a treatment regimen for dementia, the method comprising:

a) providing a whole blood sample from the subject;

b) determining the level of one or more biomarker in the whole blood sample, wherein the one or more biomarker is selected from the group consisting of clusterin, alpha-synuclein and amyloid precursor protein (APP);

c) administering a dementia treatment regimen to the subject for a time interval;

d) repeating steps a) and b) using a whole blood sample obtained from the subject after the treatment regimen of step c); and

d) comparing the level of biomarker determined in step b) to that determined in step c), and identifying that the treatment regimen has a therapeutic effect if one or more of the following is observed: there is a change in the level of clusterin after treatment; there is an increase in the level of alpha-synuclein after treatment; or there is an increase in the level of amyloid precursor protein after treatment.

In another aspect, the invention provides an in vitro method for monitoring changes in cognition in a subject having or at risk of having dementia and treating the subject for dementia, the method comprising the steps of:

i) performing the following steps:
a) providing a whole blood sample from the subject;
b) determining the level of one or more biomarker in the whole blood sample, wherein the one or more biomarker is selected from the group consisting of clusterin, alpha synuclein (AS) and amyloid precursor protein (APP);
c) comparing the level of the one or more biomarker with the level of the same biomarker in a control sample or with a pre-determined reference level for the same biomarker to identify an increase or decrease in a level of the one or more biomarker in the sample of the subject;

ii) repeating i) for the same subject after a time interval;

iii) comparing the biomarker levels identified in i) with the biomarker levels identified in ii), and identifying a reduction in cognitive score if the comparison in step iii) indicates that the subject has one or more of the following: a change in the level of clusterin over the time interval; an increase in the level of alpha-synuclein over the time interval; or a decrease in the level of amyloid precursor protein over the time interval; and

iv) administering an effective amount of at least one anti-dementia compound to a subject identified as having a reduction in cognitive score.

In another aspect, the invention provides a method of treating a subject having or at risk of having dementia, the method comprising administering an effective amount of at least one anti-dementia compound to the subject, wherein the subject has been identified as having a reduced cognitive score using a method described elsewhere herein.

In another aspect, the invention provides a method of monitoring changes in cognition in a subject having or at risk of having dementia, the method comprising:

a. obtaining a whole blood sample from a human patient and

b. detecting the level of one or more biomarker in the whole blood sample, wherein the one or more biomarker is selected from the group consisting of clusterin, alpha synuclein (AS) and amyloid precursor protein (APP), by contacting the whole blood sample with an appropriate antibody specific to the biomarker of interest and detecting binding between the biomarker and the corresponding antibody (i.e. clusterin binding to an anti-clusterin antibody; alpha synuclein (AS) binding to an anti-AS antibody or amyloid precursor protein (APP) binding to an anti-APP antibody);

c. repeating steps a and b for the same subject after a time interval;

d. comparing the biomarker levels identified in b) with the biomarker levels identified in c), wherein a change in the biomarker levels from b) to c) is indicative of a change in cognitive score in the subject; and

e. administering an effective amount of at least one anti-dementia compound to a subject identified as having a reduction in cognitive score.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Various aspects of the invention are described in further detail below.

### DETAILED DESCRIPTION

The inventors have surprisingly found new biomarkers for dementia in whole blood, namely clusterin, alpha-synuclein and amyloid precursor protein (APP). One or more (e.g. two or three) of these biomarkers can advantageously be used in any of the methods, kits, assays, or uses described herein.

Methods for Diagnosing Dementia or Determining the Risk of Developing Dementia in a Subject

In one aspect, an in vitro method for diagnosing dementia or determining the risk of developing dementia in a subject is provided, the method comprising the steps of:

a) providing a whole blood sample from the subject;

b) determining the level of one or more biomarker in the whole blood sample, wherein the one or more biomarker is selected from the group consisting of clusterin, alpha synuclein (AS) and amyloid precursor protein (APP);

c) comparing the level of the one or more biomarker with the level of the same biomarker in a control sample or with a pre-determined reference level for the same biomarker to identify an increase or decrease in a level of the one or more biomarker in the sample of the subject;

d) identifying a subject as having dementia or as having an increased risk of developing dementia if the comparison in step c) indicates that the subject has one or more of the following: a change in the level of clusterin compared to the control sample or the pre-determined reference level; an increased level of alpha-synuclein compared to the control sample or the pre-determined reference level; or a decreased level of amyloid precursor protein compared to the control sample or the pre-determined reference level.

The method is useful for diagnosing dementia or determining the risk of developing dementia in a subject. The term “subject” as used herein refers to a mammal. A subject therefore refers to, for example, dogs, cats, horses, cows, pigs, guinea pigs, and the like. The subject can be a human.

The subject may be referred to herein as a patient. The terms “subject”, “individual”, and “patient” are used herein interchangeably. The subject can be symptomatic (e.g., the subject presents symptoms associated with dementia or dementia related neurological disorders), or the subject can be asymptomatic (e.g., the subject does not present symptoms associated with dementia or dementia related neurological disorders).

The subject may be diagnosed with, be at risk of developing or present with symptoms of dementia. The subject may have, or be suspected of having (e.g. present with symptoms or a history indicative or suggestive of), dementia or a dementia-related neurological disorder as described herein.

Accordingly, in some examples, the subject has dementia or a dementia-related neurological disorder. In particular examples, the subject has early stage dementia or an early stage dementia-related neurological disorder. An example of an early stage of disease is when the subject is in the prodromal stages of the disorder, wherein they have the initial symptoms of the disorder but have not yet developed the sufficient symptoms for diagnosis of disease.

Dementia refers to a group of symptoms that is well defined in the art. As used herein, “dementia” refers broadly to any disorder, disease, or syndrome characterized by an abnormally high and progressive loss of functional capacity of the brain. While symptoms of dementia can vary greatly, hallmarks of dementia include impairment of several core mental functions, including memory, communication and language, ability to focus and pay attention, reasoning and judgment, and visual perception. Many dementias are progressive, meaning symptoms start out slowly and gradually get worse. Dementia may be determined using standard clinical procedures, with the degree of dementia being defined by the score in the Mini Mental State Examine (MMSE), as detailed in Folstein M. F., Folstein S. E. and McHugh P. R., J Psychiatry Res., 12: 189-198 (1975). For example, a score of 30 to 27 points in the MMSE is classified as non-demented, a score of 26 to 20 is considered mildly demented, a score of 19 to 10 points is considered moderately demented and a score of 9 to 0 points is considered severely demented. Dementia, as used herein, includes all ranges of scores of the MMSE, except, of course, those scores classified as non-demented. However, “dementia,” as used herein, is not to be limited by the presence or absence of an MMSE score. Other examples include the Cambridge Cognition Examination (CAMCOG), the Addenbrooke's Cognitive Assessment (ACE-III), mini ACE, the abbreviated mental test score (AMTS), the Montreal Cognitive Assessment (MoCA), the Modified Mini-Mental State Examination (3MS), the Cognitive Abilities Screening Instrument (CASI), the Trail-making test, and the clock drawing test, all of which are clearly defined in the art. All of the above are considered herein as different means for assessing cognition in a subject (i.e. assessing their cognitive score and/or assessing changes in cognition/cognitive score).

Accordingly, as outlined herein changes in cognition/changes in cognitive score can be assessed using any one of the following methods: MMSE, Cambridge Cognition Examination (CAMCOG), the Addenbrooke's Cognitive Assessment (ACE-III), mini ACE, the abbreviated mental test score (AMTS), the Montreal Cognitive Assessment (MoCA), the Modified Mini-Mental State Examination (3MS), the Cognitive Abilities Screening Instrument (CASI), the Trail-making test, and the clock drawing test, all of which are clearly defined in the art.

Symptoms of dementia in a subject may be present because of a dementia-related neurological disorder. In other words, the symptoms of dementia may be caused by an underlying dementia-related neurological disorder in the subject. The subject therefore being identified as having or at risk of having (or at risk of developing) dementia may therefore be identified as having a dementia-related neurological disorder (as the underlying cause of the dementia symptoms). As used herein, the term “dementia” encompasses “dementia-related neurological disorder” unless the context specifically indicates otherwise.

As used herein, a “dementia-related neurological disorder” is a neurological disease characterized by the presence of dementia. Examples of a dementia related neurological disorder include, but are not limited to, Alzheimer's Disease (AD), progressive supranuclear palsy (PSP), Huntington's Disease (HD), dementia of mixed type, Parkinson's Disease, diffuse Lewy Body dementia, Vascular dementia (VaD), frontotemporal dementia, semantic dementia and Dementia with Lewy Bodies (DLB).

The biomarkers provided herein are particularly useful for diagnosing or determining the risk of developing dementia e.g. wherein the dementia is caused by a dementia-related neurological disorder. Suitably, the dementia-related neurological disorder is Alzheimer's disease, Vascular dementia or DLB.

The phrase “Alzheimer's disease” as used herein refers to a progressive disease of the human central nervous system. It is manifested by dementia typically in the elderly, by disorientation, loss of memory, difficulty with language, calculation, or visual-spatial skills, and by psychiatric manifestations. It is associated with degenerating neurons in several regions of the brain. Histologically, the disease is characterized by neuritic plaques, found primarily in the association cortex, limbic system and basal ganglia. The phrase “Alzheimer's disease” as used herein is intended to encompass all types of Alzheimer's disease, including sporadic and familial AD, as well as late onset and early onset AD.

In Alzheimer's disease, the pathological protein is amyloid beta, which in humans can be identified using NCBI GenBank or UniProt (Gene ID: 351; Protein ID: P05067). “amyloid beta” or “Aβ” refers to peptides of 36-43 amino acids that are the main component of the amyloid plaques found in the brains of Alzheimer's patients. The peptides derive from the amyloid precursor protein (APP), which is cleaved by beta secretase and gamma secretase to yield Aβ. A molecules can aggregate to form flexible soluble oligomers which are toxic to nerve cells. The structure of amyloid beta is described in Schmidt et al. 2015. Aβ can be detected directly by the presence of Aβ aggregates/Aβ aggregation. Several methods for detecting aggregation of these proteins are well known including immunoelectron microscopy (EM).

Alzheimer's disease is clinically well defined, details of which can be found in Desai et al. 2005; McKhann et al. 2011; and Dubois et al. 2014.

“Vascular dementia” (VaD) is a common form of dementia. The term “Vascular dementia” refers to a group of syndromes relating to different Vascular mechanisms. Various subtypes of Vascular dementia have been described to date. The spectrum of disease includes (1) mild Vascular cognitive impairment, (2) multi-infarct dementia, (3) Vascular dementia due to a strategic single infarct, (4) Vascular dementia due to lacunar lesions, (5) Vascular dementia due to hemorrhagic lesions, (6) Binswanger disease, (7) subcortical Vascular dementia, and (8) mixed dementia (combination of AD and Vascular dementia). Vascular dementia is sometimes further classified as cortical or subcortical dementia. Vascular dementia can be diagnosed by clinical criteria, often in combination with brain imaging. More details on the clinical features and symptoms of VaD can be found in the literature, for example in US 20130156759. VaD is clinically well defined, details of which can be found in [Sachdev et al, 2014).

Dementia with Lewy bodies (DLB) is characterized by the presence of Lewy bodies (LBs) in the subcortical and cortical (frontotemporal) regions of the brain. DLB is the second most common form of dementia (Neef, et al. Am Fam Physician, 2006, 73:1223-9), at least 5% of adults 85 years or older have DLB. The clinical features of DLB include dementia (executive function deficit, visuospatial impairment), delirium, visual hallucinations, parkinsonism (bradykinesia, rigidity, tremors), and depression. LBs are also present in Parkinson's Disease (PD) patients. Therapies to treat symptoms of DLB include regulation of dopamine levels to improve mobility of DLB patients and administration of cholinesterase inhibitors to treat cognitive and behavioural problems, including visual hallucinations and delusions In dementia with Lewy bodies, the pathological protein is alpha-synuclein, which in humans can be identified using NCBI GenBank or UniProt (Gene ID: 6622; Protein ID: P37840). Dementia with Lewy bodies is clinically well defined, details of which can be found in McKeith et al. 2017.

In general, the methods described are in vitro methods that are performed using a sample that has already been obtained from the subject (i.e. the sample is provided for the method, and the steps taken to obtain the sample from the subject are not included as part of the method).

As used herein, “provide”, “obtain” or “obtaining” can be any means whereby one comes into possession of the sample by “direct” or “indirect” means. Directly obtaining a sample means performing a process (e.g., performing a physical method such as extraction) to obtain the sample. Indirectly obtaining a sample refers to receiving the sample from another party or source (e.g., a third party laboratory that directly acquired the sample).

The methods provided herein comprise providing a biological sample (specifically a whole blood sample) from a subject. The samples being tested in the methods described herein are also referred to as “test sample”.

As used herein, the term “biological sample”, “test sample” or “sample” refers to a sample obtained or derived from a subject. For the purposes described herein, the sample is, or comprises, a whole blood sample. A whole blood sample is defined as a blood sample drawn from the human body and from which (substantially) no constituents (such as platelets or plasma) have been removed. In other words, the relative ratio of constituents in a whole blood sample is substantially the same as a blood in the body. In this context, “substantially the same” allows for a very small change in the relative ratio of the constituents of whole blood e.g. a change of up to 5%, up to 4%, up to 3%, up to 2%, up to 1% etc.

Whole blood contains both the cell and fluid portions of blood. A whole blood sample may therefore also be defined as a blood sample with (substantially) all of its cellular components in plasma, wherein the cellular components (i.e. at least comprising the requisite white blood cells, red blood cells, platelets of blood) are intact.

Methods for obtaining whole blood samples from a subject are well known and include established techniques used in phlebotomy.

Typically, a whole blood sample has been withdrawn from a subject into an anticoagulant solution such as, but not limited to Ethylenediaminetetraacetic acid (EDTA).

The methods provided herein include the step of determining the level of one or more biomarker in the whole blood sample, wherein the one or more biomarker is selected from the group consisting of clusterin, alpha-synuclein and amyloid precursor protein (APP).

A biomarker is an organic biomolecule (e.g. a protein, polypeptide, peptide, isomeric form thereof, immunologically detectable fragment thereof, corresponding nucleic acid molecule (e.g. mRNA, cDNA etc) which is differentially present in a sample taken from a subject having a disease as compared with a subject not having the disease. A biomarker is differentially present if the mean or median level of the biomarker in the different groups is calculated to be statistically significant. Common tests for statistical significance include, among others, t-test (e.g., student t-test), ANOVA, Kruskal-Wallis, Wilcoxon, Mann-Whitney, Receiver Operating Characteristic (ROC curve), accuracy and odds ratio. Biomarkers, alone or in combination, provide measures of relative risk that a subject belongs to one phenotypic status or another. Therefore, they are useful as markers for disease (diagnostics), therapeutic effectiveness of a drug and drug toxicity.

Typically, the biomarker referred to herein is measured at the protein or mRNA level (either directly or indirectly, e.g. via the generation of cDNA that corresponds to the mRNA for the particular biomarker(s) of interest).

“Clusterin” (also known as Apolipoprotein J, SGP-2, TRPM-2 and SP-40) is an extracellular protein with a nearly ubiquitous tissue distribution. It is encoded by the CLU gene on chromosome 8. Despite its ubiquitous expression and its relative abundance in serum, clusterin's function remains unknown. It has been linked to the ability to inhibit complement cascade by binding C9 complement, pro-apoptotic activity or an anti-apoptotic activity depending on animal models studied, limitation of progression and chaperone properties. A neuroprotective role of clusterin in Alzheimer's disease has also been suggested. Its major form, a 75-80 kDa heterodimer, is issued from a single transcript.

Human clusterin is composed of two disulfide-linked α (34-36 kD) and β (36-39 kD) subunits derived from a single amino acid chain (449 amino acids in human) that becomes glycosylated in the endoplasmic reticulum and Golgi bodies and undergoes intramolecular cleavage and dimerization before secretion. The first 22 amino acids comprise the secretory signal sequence. The cleavage site between the α and β chains is between amino acids 227 and 228. Clusterin contains three hydrophobic domains, a long α-helix motif near the amino terminal and at least six N-linked glycosylation sites. Clusterin also contains a hemopexin-like domain at the C-terminus of the enzyme, which modulates the processing and activity of the enzymes by serving as a binding region for regulatory or target proteins. Human clusterin can be identified using UniProt (Protein ID: E7ERK6).

“Alpha-synuclein” (AS or α-synuclein, also known as SNCA, NACP, PARK1, PARK4, PD1, synuclein alpha) is a synuclein protein of unknown function primarily found in neural tissue. Recently, the alpha-synuclein has been identified in different components of blood, including erythrocytes (ERC). AS is most commonly found at the synaptic ends of neurons within the brain and is encoded by SCNA gene on chromosome 4. Currently, its role is not yet fully understood; however studies have shown involvement in vesicle trafficking and neurotransmitter metabolism. AS is comprised of 140 amino acids and is arranged natively as an unfolded protein of 14 kDa, but when bound to lipid vesicles adopts a helical folded structure. It has a repeated sequence of KTKEGV situated at the N-terminal which is thought to aid the binding to other molecules and proteins. In the brain tissue of DLB subjects, AS is found within intraneuronal aggregates called Lewy bodies that cause neuronal damage leading to neuronal death and a decline in brain mass. Lewy Bodies are most commonly found in DLB, Parkinson's disease, and other synucleinopathies, in which they accumulate in the Substantia nigra causing damage to the pars compacta and resulting in subsequent loss of dopaminergic neurons. Additionally, Lewy bodies have also been discovered in the neurons of other types of dementia such as Alzheimer's disease (AD), with up to 50% of late-onset AD cases having AS aggregates in their brain tissue, and are also present in cognitively intact older people (in up to 26% of older healthy subjects, and are not related to ageing or incidental presence of AD pathology). Human AS can be identified using NCBI GenBank or UniProt (Gene ID: 6622; Protein ID: P37840).

“Amyloid precursor protein” (APP, also known as APP, AAA, ABETA, ABPP, AD1, APPI, CTFgamma, CVAP, PN-II, PN2, amyloid beta precursor protein and preA4) is a single-pass transmembrane protein expressed in many tissues, and found especially concentrated in the brain, specifically in the synapses of neurons. It's gene maps on chromosome 21. While its primary function is not known, it has been implicated as a regulator of synapse formation, neural plasticity and iron export. APP is best known as the precursor molecule whose proteolysis generates beta amyloid (Aβ), a polypeptide containing 37 to 49 amino acid residues, whose amyloid fibrillar form is the primary component of amyloid plaques found in the brains of Alzheimer's disease patients. Genetic, biochemical, and behavioural research suggest that physiologic generation of the neurotoxic Aβ peptide from sequential APP proteolysis is a crucial step in the development of AD. APP metabolism is highly complex and changes in APP metabolism or Aβ elimination could possibly lead to AD.

The secreted ectodomain fragment of APP (sAPPα) can be readily cleaved to produce a small N-terminal fragment (APP-N). This fragment contains heparin-binding and metal-binding domains, and has been found to have biological activity. APP-N can bind to the extracellular surface of neurons and glia, and may mediate the activity of APP. In the studies described herein, a polyclonal antibody (A8967 Sigma) against the N-terminal end of APP was used to detect APP. The antibody was raised against a synthetic peptide corresponding to the N-terminal of human APP695, corresponding to 46-40 amino acids of the APP protein. The antibody thus immunolabels APP isoforms APP770, APP751, and APP 605. In humans, APP can be identified using NCBI GenBank or UniProt (Gene ID: 351; UniProtKB (protein ID)-P05067).

In one example, the level of APP is determined by measuring the level of APP-N in the whole blood sample. Measurement of APP-N would detect the presence of total APP as APP cleavage in vivo (via alpha, beta and gamma secretases) occurs towards the C-terminal portion of the APP molecule that contains the amyloid beta peptide. Thus, the N terminal end of the APP is not affected by the secretases. Accordingly, detection of APP-N corresponds to detection of both full-length APP and the soluble APP fragment.

Conventional “determining” methods may include sending a clinical sample(s) to a commercial laboratory for measurement of the biomarker levels in the whole blood sample, or the use of commercially available assay kits for measuring the biomarker levels in the whole blood sample. Exemplary kits and suppliers will be apparent to the skilled artisan. In various examples, biomarkers may be determined, detected and/or quantified using lateral flow devices, such as for point-of-care use, as well as spot check colorimetric tests.

The level of biomarker present in the whole blood sample may be determined by e.g. assaying the amount of protein biomarker present in the sample, or e.g. the amount of mRNA present in the sample. Assays for measuring the amount of a specified protein or mRNA are well known in the art and include direct or indirect measures (e.g. detecting cDNA as an indirect measure of the amount of mRNA present within a sample).

The level of protein biomarker in a sample may also be determined by determining the level of protein biomarker activity in a sample. Accordingly, protein level encompasses both the amount of protein per se, or its level of activity.

By way of example, the level of a protein biomarker in a whole blood sample can be determined (e.g., measured) by any suitable methods and materials known in the art, including, for example, a process selected from the group consisting of mass spectrometry, immunoassays, enzymatic assays, spectrophotometry, colorimetry, fluorometry, bacterial assays, protein microarrays, compound separation techniques, or other known techniques for determining the presence and/or quantity of an analyte. Examples of relevant techniques include enzyme linked immunosorbent assays (ELISAs), immunoprecipitation, immunofluorescence, enzyme immunoassay (EIA), radioimmunoassay (RIA), Western blot analysis, and Lateral Flow (using e.g. Lateral Flow Devices (LFDs) utilizing a membrane bound antibody specific to the protein biomarker). Preferably, the level of a protein biomarker in a whole blood sample is measured by ELISA or lateral flow.

By way of example, the level of an mRNA biomarker can be determined (e.g., measured) by any suitable methods and materials known in the art, including, for example, microarray analysis or reverse transcription PCR (RT-PCR). Such methods are routine in the art.

In an example, the methods described herein determine the level of two or three of the specified biomarkers. For example, the method may determine the level of clusterin and AS; clusterin and APP; AS and APP; or clusterin, AS and APP.

Methods described herein further comprise comparing the level of the at least one biomarker (i.e. its amount per se or its activity) in the whole blood sample (“test sample”) with the level of the at least one biomarker in a control sample or with a predetermined reference level for the at least one biomarker.

In one example, methods described may therefore include contacting a control whole blood sample with a compound or agent capable of detecting a specific biomarker mRNA (e.g. clusterin mRNA, AS mRNA or APP mRNA), and comparing the level of the biomarker mRNA in the control sample with the level of biomarker mRNA in the test sample.

In another example, the methods described may include contacting a control sample with a compound or agent capable of detecting a specific biomarker protein (e.g. clusterin protein, AS protein or APP protein), and comparing the level of the biomarker protein in the control sample with the presence of the biomarker protein in the test sample.

As used herein “control sample”, refers to a sample having a normal level of biomarker (e.g. clusterin, AS or APP), for example a sample obtained in at least one individual not suffering from dementia or a dementia related neurological disorder from the same species. Such individuals are referred to herein as “non-demented”. The individual can be the same age, sex or in the same state or condition of health as the subject from which the test sample is obtained.

The control sample may be assayed at the same time, before or after, separately or simultaneously with the test sample. The control value that is used in the comparison with the test sample may be a value that is calculated as an average or median of more than one (e.g. two or more, five or more, ten or more, a group etc) of control samples. Alternatively, the control sample may be a sample that originated from (i.e. is a mix of) more than one (e.g. two or more, five or more, ten or more, a group etc) individual that is not suffering from dementia or a dementia related neurological disorder.

In one example, the control sample is therefore obtained from a non-demented control subject.

Alternatively, the level of biomarker (protein or mRNA) in the whole blood sample may be compared to a pre-determined reference level for the biomarker of interest.

As used herein, a “predetermined reference level” refers to a biomarker level obtained from a reference database, which may be used to generate a pre-determined cut off value, i.e. a score that is statistically predictive of dementia or a dementia-related neurological disorder. In one example, the predetermined reference level is the average or median level of the biomarker in at least one individual not suffering from dementia or a dementia related neurological disorder from the same species. Such individuals are referred to herein as “non-demented”. The predetermined reference value may be calculated as the average or median, taken from a group or population of individuals that are not suffering from dementia or a dementia related neurological disorder. The individual or the population of individuals can be the same age, sex or in the same state or condition of health as the subject from which the test sample is obtained.

In one example, the pre-determined reference level is therefore the average level of the biomarker in a non-demented control subject.

Typically, in methods for diagnosing dementia or determining the risk of developing dementia in a subject, the control sample or predetermined reference are obtained from an individual or group of individuals that are distinct from the subject that is being tested (i.e. the subject from which the test sample is obtained/provided). In such examples, the control or predetermined reference are used as a bench line to determine whether the tested subject has or is at risk of having dementia.

In an alternative example, the control or predetermined reference value may be obtained from the same individual as the test sample, but at an earlier time point. This is particularly relevant for the methods described herein that monitor dementia progression in a subject, determine the therapeutic effect of a treatment regimen for dementia, determine a subject's compliance or adherence with a prescribed treatment regimen for dementia, and/or monitor cognitive performance (with clinical cognitive tests, such as CAMCOG and/or MMSE and/or other cognitive clinical tests, i.e. MoCA, ACE-Ill, Mini ACE etc.) scores in a subject having or at risk of having dementia. In such examples, the control sample or predetermined reference level is used to determine any changes in the level of the biomarker(s) over a time interval for the same subject.

The pre-determined reference level or control sample can therefore be from the same subject that the test sample is obtained from, for example obtained at an earlier time point. This earlier time point can be before they were diagnosed with or known to be at risk of developing dementia or a dementia related neurological disorder.

A pre-determined level can be single cut-off value, such as a median or mean. It can be a range of cut-off (or threshold) values, such as a confidence interval. It can be established based upon comparative groups, such as where the risk in one defined group is a fold higher, or lower, (e.g., approximately 2-fold, 4-fold, 8-fold, 16-fold or more) than the risk in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk. Moreover, the reference could be a calculated reference, most preferably the average or median, for the relative or absolute amount of a biomarker of a population of individuals comprising the subject to be investigated. How to calculate a suitable reference value, preferably, the average or median, is well known in the art. The population of subjects referred to before shall comprise a plurality of individuals, preferably, at least 5, 10, 50, 100, 1,000 subjects.

Thus, in some cases the level of the protein biomarker in a subject being greater than or equal to the level of the biomarker of the control sample or pre-determined reference level is indicative of a clinical status (e.g., indicative of a dementia or a dementia related neurological disorder diagnosis). In other cases the level of the biomarker in a subject being less than or equal to the level of biomarker of the control sample or predetermined reference level is indicative of a clinical status. The amount of the greater than and the amount of the less than is usually of a sufficient magnitude to, for example, facilitate distinguishing a subject from a control subject using the disclosed methods. Typically, the greater than, or the less than, that is sufficient to distinguish a subject from a control subject is a statistically significant greater than, or a statistically significant less than. In cases where the level of the biomarker in a subject being equal to the level of the biomarker in a control subject is indicative of a clinical status, the “being equal” refers to being approximately equal (e.g., not statistically different).

The pre-determined value can depend upon a particular population of subjects (e.g., human subjects) selected. For example, an apparently healthy population will have a different ‘normal’ range of the protein biomarker than will a population of subjects which have, or are likely to have, a dementia or a dementia related neurological disorder. Accordingly, the pre-determined values selected may take into account the category (e.g., healthy, at risk, diseased) in which a subject (e.g., human subject) falls. Appropriate ranges and categories can be selected with no more than routine experimentation by those of ordinary skill in the art.

Suitably, the level of the specific biomarker detected in a sample (e.g. a test sample, a control sample etc) is normalized by adjusting the measured level (amount or activity) of the biomarker using the level of a reference mRNA or protein (as appropriate) in the same sample, wherein the reference mRNA or protein is not a marker itself (it is e.g., an mRNA or protein that is constitutively expressed). This normalization allows the comparison of the biomarker level in one sample to another sample, or between samples from different sources. This normalized level can then optionally be compared to a reference value or control.

For example, when measuring a protein biomarker in a whole blood sample the biomarker may be expressed as an absolute concentration or, alternatively, it may be normalized against a known protein constitutively expressed in whole blood such as albumin, immunoglobulins or plasma protein concentration.

The biomarker level(s) in the test sample may be compared to the level of the same biomarker in a control sample or with a pre-determined reference level for the same biomarker to identify an increase or decrease in a level of the one or more biomarker in the sample of the subject.

In the methods described herein, the subject may be identified as having dementia or as having an increased risk of developing dementia if the comparison (between biomarker level(s) in the control sample/predetermined reference value and the test sample of the subject) indicates that the subject has one or more of the following: a change in the level of clusterin compared to the control sample or the pre-determined reference level; an increased level of alpha-synuclein compared to the control sample or the pre-determined reference level; or a decreased level of amyloid precursor protein compared to the control sample or the pre-determined reference level.

In a particular example, the subject may be identified as having Alzheimer's disease or as having an increased risk of developing Alzheimer's disease when they have a decreased level of clusterin in their whole blood sample compared to the control sample or the pre-determined reference level (e.g. when the level of clusterin (protein or mRNA) in their whole blood sample is lower than the level of clusterin (protein or mRNA respectively) in the control sample or predetermined reference sample that has been obtained from a non-demented individual or individuals).

In another particular example, the subject may be identified as having Vascular dementia or as having an increased risk of developing Vascular dementia when they have an increased level of clusterin in their whole blood sample compared to the control sample or the pre-determined reference level (e.g. when the level of clusterin (protein or mRNA) in their whole blood sample is higher than the level of clusterin (protein or mRNA respectively) in the control sample or predetermined reference sample that has been obtained from a non-demented individual or individuals).

The term “change” refers in this context to a statistically significant difference in the biomarker level for the sample obtained from the test subject compared to the biomarker levels obtained from the control sample or predetermined reference level. The difference (or change) may be an increase or decrease in biomarker levels compared to the control sample or predetermined reference level.

The terms “decrease”, “decreased” “reduced”, “reduction” or ‘down-regulated”, “lower” are all used herein generally to mean a decrease by a statistically significant amount. However, for avoidance of doubt, “reduced”, “reduction”, “decreased” or “decrease” means a decrease by at least 10% as compared to a reference level/control, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference/control sample), or any decrease between 10-100% as compared to a reference level/control, or at least about a 0.5-fold, or at least about a 1.0-fold, or at least about a 1.2-fold, or at least about a 1.5-fold, or at least about a 2-fold, or at least about a 3-fold, or at least about a 4-fold, or at least about a 5-fold or at least about a 10-fold decrease, or any decrease between 1.0-fold and 10-fold or greater as compared to a reference level/control.

The terms “increased”, “increase” or “up-regulated”, “higher” are all used herein to generally mean an increase by a statically significant amount; for the avoidance of any doubt, the terms “increased” or “increase” means an increase of at least 10% as compared to a reference level/control, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level/control, or at least about a 0.5-fold, or at least about a 1.0-fold, or at least about a 1.2-fold, or at least about a 1.5-fold, or at least about a 2-fold, or at least about a 3-fold, or at least about a 4-fold, or at least about a 5-fold or at least about a 10-fold increase, or any increase between 1.0-fold and 10-fold or greater as compared to a reference level/control.

The methods can further comprise selecting, and optionally administering, a treatment for the subject based on the diagnosis (i.e., based on the comparison of the levels of the biomarkers with the reference levels/controls). The treatment can include, for example, administering to the subject an effective amount of at least one anti-dementia compound (also known as a therapeutic agent, medicament or composition herein).

As used herein, the terms “treat”, “treating” and “treatment” are taken to include an intervention performed with the intention of preventing the development or altering the pathology of a condition, disorder or symptom (i.e. in this case dementia, and more specifically dementia-related neurological disorders such as AD, VaD and DLB). Accordingly, “treatment” refers to both therapeutic treatment and prophylactic or preventative measures, wherein the object is to prevent or slow down (lessen) the targeted condition, disorder or symptom. “Treatment” therefore encompasses a reduction, slowing or inhibition of the symptoms of dementia [e.g. as measured by CAMCOG, MMSE, ACE-Ill, MoCA or other cognitive assessment tool values, and Behavioural and Psychological Symptoms of Dementia (BPSD) as measured with the Neuropsychiatric Inventory (NPI) or other similar behavioural assessment tools), for example of at least 5%, 10%, 20%, 30%, 40%, 50%, 60%, 70%, 80%, 90% or 100% when compared to the symptoms before treatment.

Anti-dementia compounds are well known in the art and some are disclosed herein. Non-limiting examples include donepezil, rivastigmine, galantamine, memantine, tacrine, or salts thereof.

The type of treatment will vary depending on the particular form of dementia or dementia-related neurological disorder that that the subject has, is suspected of having, is at risk of developing, or is suspected of being at risk of developing.

For example, if the subject has, is suspected of having, is at risk of having, or is suspected of being at risk of having, dementia with Lewy bodies, the subject may benefit from treatment with for example cholinesterase inhibitors—such as donepezil or rivastigmine Accordingly, the method may include the step of administering cholinesterase inhibitors to the subject or other anti-dementia drugs that are still under development. Other suitable treatments are well known to a person of skill in the art and depend on the specific symptoms of the subject.

As a further example, if the subject has, is suspected of having, is at risk of having, or is suspected of being at risk of having, Alzheimer's disease, the subject may benefit from treatment with cholinesterase inhibitors, such as donepezil, rivastigmine and galantamine and NMDA antagonist, such as memantine. Accordingly, the method may include the step of administering cholinesterase inhibitors and/or NMDA antagonist to the subject. Other suitable treatments are well known to a person of skill in the art and depend on the specific symptoms of the subject.

As a further example, if the subject has, is suspected of having, is at risk of having, or is suspected of being at risk of having, Vascular dementia, the subject may benefit from treatment with cholinesterase inhibitors—such as doinepezil, rivastigmine and galantamine and or NMDA antagonist, such as memantine. Accordingly, the method may include the step of administering cholinesterase inhibitors and/or NMDA antagonist to the subject. Other suitable treatments are well known to a person of skill in the art and depend on the specific symptoms of the subject.

When a therapeutic agent or other treatment is administered, it is administered in an amount effective to treat dementia or a dementia related neurological disorder or to reduce the likelihood (or risk) of future dementia or dementia related neurological disorder developing. An effective amount is a dosage of the therapeutic agent sufficient to provide a medically desirable result. The effective amount will vary with the particular condition being treated, the age and physical condition of the subject being treated, the severity of the condition, the duration of the treatment, the nature of the concurrent therapy (if any), the specific route of administration and the like factors within the knowledge and expertise of the health care practitioner. For example, an effective amount can depend upon the degree to which a subject has abnormal levels of certain analytes (e.g., biomarkers as described herein) that are indicative of dementia or a dementia related neurological disorder. It should be understood that the therapeutic agents described herein are used to treat and/or prevent dementia or a dementia related neurological disorder. Thus, in some cases, they may be used prophylactically in subjects at risk of developing dementia or a dementia related neurological disorder. Thus, in some cases, an effective amount is that amount which can lower the risk of, slow or perhaps prevent altogether the development of a dementia or a dementia related neurological disorder. It will be recognized when the therapeutic agent is used in acute circumstances, it is used to prevent one or more medically undesirable results that typically flow from such adverse events. Methods for selecting a suitable treatment, an appropriate dose thereof and modes of administration will be apparent to one of ordinary skill in the art.

The medications (also referred to as compositions or compounds e.g. anti-dementia compounds herein) described herein can be administered to the subject by any conventional route, including injection or by gradual infusion over time. The administration may, for example, be by infusion or by intramuscular, intravascular, intracavity, intracerebral, intralesional, rectal, subcutaneous, intradermal, epidural, intrathecal, percutaneous administration. The medications may also be given in e.g. tablet form or in solution.

The compositions described herein may be in any form suitable for the above modes of administration. For example, suitable forms for parenteral injection (including, subcutaneous, intramuscular, intravascular or infusion) include a sterile solution, suspension or emulsion; suitable forms for topical administration include an ointment or cream; and suitable forms for rectal administration include a suppository. Alternatively, the route of administration may be by direct injection into the target area, or by regional delivery or by local delivery. The identification of suitable dosages of the compositions of the invention is well within the routine capabilities of a person of skill in the art.

The composition is preferably for, and therefore formulated to be suitable for, administration to a subject, preferably a human or animal subject. Preferably, the administration is parenteral, e.g. intravenous, subcutaneous, intramuscular, intradermal, intracutaneous and/or intrathecaladministration, i.e. by injection.

Kits and Assay Devices

In another aspect, kits are provided for diagnosing dementia or determining the risk of developing dementia in a subject. The kits include reagents suitable for determining levels of a plurality of analytes in a test sample (e.g., reagents suitable for determining levels of the biomarkers disclosed herein).

The kits described herein typically comprise:

(i) a detectably labelled agent that specifically binds to clusterin; and

(ii) one or more of:
a) a detectably labelled agent that specifically binds to alpha-synuclein; and
b) a detectably labelled agent that specifically binds to amyloid precursor protein (APP).

The kits may alternatively comprise a detectably labelled agent that specifically binds to alpha-synuclein and a detectably labelled agent that specifically binds to amyloid precursor protein (APP).

In some examples, the kit comprises a detectably labelled agent that specifically binds to alpha-synuclein, a detectably labelled agent that specifically binds to amyloid precursor protein (APP), and a detectably labelled agent that specifically binds to clusterin.

The kits described herein can take on a variety of forms. Typically, the kits will include reagents suitable for determining levels of a plurality of biomarkers (e.g., those disclosed herein, for example clusterin and AS, clusterin and APP, AS and APP, or clusterin, AS and APP) in a sample.

Optionally, the kits may contain one or more control samples or references. Typically, a comparison between the levels of the biomarkers in the subject and levels of the biomarkers in the control samples is indicative of a clinical status (e.g., diagnosis of dementia or risk of developing dementia etc.). Also, the kits, in some cases, will include written information (indicia) providing a reference (e.g., pre-determined values), wherein a comparison between the levels of the biomarkers in the subject and the reference (pre-determined values) is indicative of a clinical status (e.g., diagnosis of dementia or risk of developing dementia etc.). In some cases, the kits comprise software useful for comparing biomarker levels or occurrences with a reference (e.g., a prediction model). Usually the software will be provided in a computer readable format such as a compact disc, but it also may be available for downloading via the internet. However, the kits are not so limited and other variations with will apparent to one of ordinary skill in the art.

The components of the kit may be housed in a container that is suitable for transportation. Details on the biomarkers is given above and apply equally here. Suitably, the biomarker may be protein or mRNA.

The term “detectably labelled agent” refers to a binding partner that interacts (i.e. binds) specifically with the biomarker of interest [i.e. clusterin, AS or APP (e.g. APP-N)] and is also capable of being detected e.g. directly (such as via a fluorescent tag) or indirectly (such as via a labelled secondary antibody). The detectably labelled agent is therefore a selective binding partner for the biomarker of interest (and does not substantially bind to other proteins). Selective binding partners may include antibodies that selectively bind to one of the biomarker of interest.

As used herein, “specifically binds to clusterin” refers to selective binding of the clusterin peptide (or mRNA as appropriate). Under certain conditions, for example in an immunoassay as described herein, a binding partner that “specifically binds to clusterin” will selectively bind to this peptide and will not bind in a significant amount to other peptides. Thus the binding partner may bind to clusterin with at least 10, 20, 30, 40, 50, 60, 70, 80, 90 or 100 fold more affinity than it binds to a control peptide.

As used herein, “specifically binds to AS” refers to selective binding of the AS peptide (or mRNA as appropriate). Under certain conditions, for example in an immunoassay as described herein, a binding partner that “specifically binds to AS” will selectively bind to this peptide and will not bind in a significant amount to other peptides. Thus the binding partner may bind to AS with at least 10, 20, 30, 40, 50, 60, 70, 80, 90 or 100 fold more affinity than it binds to a control peptide.

As used herein, “specifically binds to APP” refers to selective binding of the APP peptide (or mRNA as appropriate). Under certain conditions, for example in an immunoassay as described herein, a binding partner that “specifically binds to APP” will selectively bind to this peptide and will not bind in a significant amount to other peptides. Thus the binding partner may bind to APP with at least 10, 20, 30, 40, 50, 60, 70, 80, 90 or 100 fold more affinity than it binds to a control peptide.

In some examples the kits include the detectably labelled agent(s) on a continuous (e.g. solid) surface, such as a lateral flow surface. Alternatively, in examples comprising more than one detectably labelled agent, the detectably labelled agent(s) may be located in distinct (i.e. spatially separate) zones on a (e.g. solid) surface, such as a multiwall micro-titre plate (e.g. for an ELISA assay). Other appropriate surfaces and containers that are well known in the art may also form part of the kits described herein.

In one example, the kit further comprises one or more reagents for detecting the detectably labelled agent. Suitable reagents are well known in the art and include but are not limited to standard reagents and buffers required to perform any one of the appropriate detection methods that may be used (and are well known in the art). In one example, the kit comprises one or more of the following: a multi-well plate, ball bearing(s), extraction buffer, extraction bottle and a lateral flow device lateral flow device.

An assay device is also provided for diagnosing dementia or determining the risk of developing dementia in a subject.

Typically, the device comprises a surface with at least two detectably labelled agents located thereon, wherein the at least two detectably labelled agents are:

(i) a detectably labelled agent that specifically binds to clusterin; and

(ii) one or more of:
a) a detectably labelled agent that specifically binds to alpha-synuclein; and
b) a detectably labelled agent that specifically binds to amyloid precursor protein (APP).

The device may comprise a detectably labelled agent that specifically binds to clusterin, a detectably labelled agent that specifically binds to alpha-synuclein and a detectably labelled agent that specifically binds to amyloid precursor protein (APP).

The device may alternatively comprise a surface with at least two detectably labelled agents located thereon, wherein the at least two detectably labelled agents are:

(i) a detectably labelled agent that specifically binds to AS; and

(ii) one or more of:
a detectably labelled agent that specifically binds to clusterin; and
a detectably labelled agent that specifically binds to amyloid precursor protein (APP).

The device may alternatively comprise a surface with at least two detectably labelled agents located thereon, wherein the at least two detectably labelled agents are:

(i) a detectably labelled agent that specifically binds to APP; and

(ii) one or more of:
a detectably labelled agent that specifically binds to AS; and
a detectably labelled agent that specifically binds to amyloid precursor protein clusterin.

The at least two detectably labeled agents may be located in separate zones on the surface. In other words, the at least two detectably labelled agents may be located in distinct (i.e. spatially separate) zones on a (e.g. solid) surface, such as a multiwell micro-titre plate.

Detectably labelled agent(s) that specifically bind to the biomarker(s) of interest are described in detail elsewhere herein.

The assay device comprises a surface upon which the detectably labelled agents are located. Appropriate surfaces include a continuous (e.g. solid) surface, such as a lateral flow surface, a dot blot surface, a dipstick surface or a surface suitable for performing surface plasmon resonance. Other appropriate surfaces include microtitre plates, multi-well plates etc. Other appropriate surfaces that are well known in the art may also form part of the assay device described herein.

Appropriate assay device formats therefore include but are not limited to device formats suitable for performing any one of lateral flow, dot blot, ELISA, or surface plasmon resonance assays for detecting the presence, level or absence of the biomarker of interest.

Uses

Also provided herein is the use of one or more biomarkers selected from the group consisting of clusterin, alpha-synuclein and amyloid precursor protein (APP) as a whole blood biomarker for dementia.

Optionally, the cause of the dementia is a dementia-related neurological disorder, such as Alzheimer's disease (AD), Vascular dementia (VaD) or Dementia with Lewy bodies (DLB).

Details of the biomarkers, samples, methods, subjects, types of dementia etc are provided elsewhere and apply equally to this aspect.

Methods for Monitoring Dementia Progression

An in vitro method for monitoring dementia progression in a subject is also provided, the method comprising the steps of:

i) determining the level of one or more biomarker in a whole blood sample from the subject in accordance with method described above; and

ii) repeating step i) for the same subject after a time interval;

iii) comparing the biomarker levels identified in i) with the biomarker levels identified in ii), wherein a change in the biomarker levels from i) to ii) is indicative of a change in dementia progression in the subject.

The method may be used to monitor the progression of a dementia related neurological disorder such as AD, VaD or DLB, amongst others.

Typically, such monitoring methods are performed on subjects that have not yet been treated for dementia with an anti-dementia compound (i.e. they are drug naïve subjects in respect of medication that is specifically administered for the treatment of the dementia that is being monitored). Such subjects are described as “drug naïve” herein.

Monitoring the progression of dementia (and specifically a dementia related neurological disorder such as AD, VaD or DLB) in a subject over time assists in the earliest possible identification of disease progression (e.g. a worsening in disease status or disease symptoms). Such monitoring naturally involves the taking of repeated samples over time. The method may therefore be repeated at one or more time intervals for a particular subject and the results compared to monitor the development, progression or improvement in the dementia (and specifically of a dementia related neurological disorder such as AD, VaD or DLB) of that subject over time, wherein a change in the amount of level of the one or more biomarker tested for in the whole blood sample is indicative of a change in the progression of the dementia (and specifically a dementia related neurological disorder such as AD, VaD or DLB) in the subject.

Disease progression (e.g. dementia progression, particularly the progression of a dementia-related neurological disorder such as AD, VaD or DLB) may be indicated by an increase in the level of alpha-synuclein (AS) detected over time when the results of two or more time intervals are compared for the same subject. In other words, if the method is performed a plurality of times, disease progression may be indicated when the level of AS detected at the later time interval(s) is higher than that detected at the earlier time interval(s). An “increase” in the level of AS encompasses detection of AS at a later time interval when no AS was detected (i.e. it was not present at detectable levels) when the method was performed previously (i.e. at an earlier time interval) on the same subject (and an equivalent whole blood sample type). This is particularly relevant when monitoring the progression of AD or VaD in anti-dementia drug naïve subjects.

Disease progression (e.g. dementia progression, particularly the progression of a dementia-related neurological disorder such as AD, VaD or DLB) may be indicated by a decrease in the level of APP (e.g. APP-N) detected over time when the results of two or more time intervals are compared for the same subject. In other words, if the method is performed a plurality of times, disease progression may be indicated when the level of APP detected at the later time interval(s) is lower than that detected at the earlier time interval(s). An “decrease” in the level of APP encompasses no detection of APP (i.e. it is not present at detectable levels) at a later time interval when APP was detected when the method was performed previously (i.e. at an earlier time interval) on the same subject (and an equivalent whole blood sample type). This is particularly relevant when monitoring the progression of AD or VaD in anti-dementia drug naïve subjects.

Disease progression (e.g. dementia progression, particularly the progression of a dementia-related neurological disorder such as AD, VaD or DLB) may be indicated by a change in the level of clusterin detected over time when the results of two or more time intervals are compared for the same subject. In other words, if the method is performed a plurality of times, disease progression may be indicated when the level of clusterin detected at the later time interval(s) is different than that detected at the earlier time interval(s).

In this context, for AD disease progression specifically (e.g. in anti-dementia drug naïve AD subjects), disease progression may be indicated by a decrease in the level of clusterin detected over time when the results of two or more time intervals are compared for the same subject. In other words, if the method is performed a plurality of times, disease progression may be indicated when the level of clusterin detected at the later time interval(s) is lower than that detected at the earlier time interval(s). An “decrease” in the level of clusterin encompasses no detection of clusterin (i.e. it is not present at detectable levels) at a later time interval when clusterin was detected when the method was performed previously (i.e. at an earlier time interval) on the same subject (and an equivalent whole blood sample type).

Conversely, for VaD disease progression specifically (e.g. in anti-dementia drug naïve AD subjects), disease progression may be indicated by an increase in the level of clusterin detected over time when the results of two or more time intervals are compared for the same subject. In other words, if the method is performed a plurality of times, disease progression may be indicated when the level of clusterin detected at the later time interval(s) is higher than that detected at the earlier time interval(s). An “increase” in the level of clusterin encompasses detection of clusterin at a later time interval when no clusterin was detected (i.e. it was not present at detectable levels) when the method was performed previously (i.e. at an earlier time interval) on the same subject (and an equivalent whole blood sample type).

Suitable time intervals for monitoring disease progression can easily be identified by a person of skill in the art and will depend on the specific form of dementia (e.g. dementia symptom or dementia related neurological disorder e.g. AD, VaD or DLB) being monitored. As a non-limiting example, the method may be repeated at least every six months, or at least every year, or whenever clinically needed, i.e. in case of a significant change in cognitive and/or behavioural symptoms a person with dementia has.

Methods for Determining the Therapeutic Effect of a Treatment Regimen for Dementia

An in vitro method for determining the therapeutic effect of a treatment regimen for dementia is also provided, the method comprising:

a) providing a whole blood sample from the subject;

b) determining the level of one or more biomarker in the whole blood sample, wherein the one or more biomarker is selected from the group consisting of clusterin, alpha-synuclein and amyloid precursor protein (APP);

c) repeating steps a) and b) using a whole blood sample obtained from the subject after treatment for a time interval; and

d) comparing the level of biomarker determined in step b) to that determined in step c), and identifying that the treatment regimen has a therapeutic effect if one or more of the following is observed: there is a change in the level of clusterin after treatment; there is an increase in the level of alpha-synuclein after treatment; or there is an increase in the level of amyloid precursor protein after treatment.

Steps a) to c) may first be performed in accordance with the method using a whole blood sample that was obtained from the subject at a time point before the treatment regimen for dementia began. Alternatively, steps a) to c) may first be performed using a whole blood sample that was obtained from the subject at the same time as commencing the treatment regimen, or at a time point after the treatment regimen for dementia began. The method can therefore be used to determine the therapeutic effect of a treatment regimen for dementia from the outset (i.e. from the start of the regimen) or from a time point after the treatment regimen has started (i.e. determining the therapeutic effect of a treatment regimen for dementia during the treatment regimen itself).

The method can also be useful as a screening tool for determining if specific regimens or treatment modalities have a therapeutic effect on dementia. The tested regimens or treatment modalities may be new regimens or treatment modalities, modified regimens or treatment modalities, or known regimens or treatment modalities that need further testing. In this context, a treatment modality is e.g. a drug or medicament that is useful or suspected to be useful in the treatment of dementia (i.e. an anti-dementia compound as described elsewhere herein).

Typically, the cause of the dementia is a dementia-related neurological disorder such as Alzheimer's disease (AD), Vascular dementia (VaD) or Dementia with Lewy Body (DLB).

Appropriate subjects, treatments, terminology and permutations or combinations of features have been described in detail above.

A treatment regimen may be identified as having a therapeutic effect if it results in a delay in disease progression or a delay in the development of symptoms (e.g. over a treatment period). A treatment regimen may also be identified as having a therapeutic effect if it results in an improvement in disease status or symptoms (e.g. over a treatment period). Methods for determining if the treatment regimen has a therapeutic effect are well known in the art.

A treatment period refers to a time interval over which treatment occurs (e.g. 1 month, 3 months, 6 months, 1 year, 2 years, in case of a significant change in cognitive and behavioural symptoms in the course of dementia etc).

As an example, an improvement in disease status or symptoms (e.g. over a treatment period) (e.g. improvement in dementia status or symptoms, particularly the disease status or symptoms of a dementia-related neurological disorder such as AD, VaD or DLB) may be indicated by an increase in the level of alpha-synuclein (AS) detected over time when the results of two or more time intervals are compared for the same subject. In other words, if the method is performed a plurality of times, an improvement in disease status may be indicated when the level of AS detected at the later time interval(s) is higher than that detected at the earlier time interval(s). An “increase” in the level of AS encompasses detection of AS at a later time interval when no AS was detected (i.e. it was not present at detectable levels) when the method was performed previously (i.e. at an earlier time interval) on the same subject (and an equivalent whole blood sample type). This is particularly relevant when determining the therapeutic effect of a treatment regimen for subjects with AD or VaD.

An improvement in disease status or symptoms (e.g. over a treatment period) may also be indicated by stabilised levels of AS over time (compared to the level of AS observed in the absence of treatment over the equivalent time period, or compared to equivalent controls). This is particularly relevant when determining the therapeutic effect of a treatment regimen for subjects with AD or VaD.

As a further example, an improvement in disease status or symptoms (e.g. over a treatment period) (e.g. improvement in dementia status or symptoms, particularly the disease status or symptoms of a dementia-related neurological disorder such as AD, VaD or DLB) may be indicated by an increase in the level of APP (e.g. APP-N) detected over time when the results of two or more time intervals are compared for the same subject. In other words, if the method is performed a plurality of times, an improvement in disease status may be indicated when the level of APP detected at the later time interval(s) is higher than that detected at the earlier time interval(s). An “increase” in the level of APP encompasses detection of APP at a later time interval when no APP was detected (i.e. it was not present at detectable levels) when the method was performed previously (i.e. at an earlier time interval) on the same subject (and an equivalent whole blood sample type). This is particularly relevant when determining the therapeutic effect of a treatment regimen for subjects with AD or VaD.

An improvement in disease status or symptoms (e.g. over a treatment period) may also be indicated by stabilised levels of APP over time (compared to the level of APP observed in the absence of treatment over the equivalent time period, or compared to equivalent controls). This is particularly relevant when determining the therapeutic effect of a treatment regimen for subjects with AD or VaD.

As a further example, an improvement in disease status or symptoms (e.g. over a treatment period) (e.g. improvement in dementia status or symptoms, particularly the disease status or symptoms of a dementia-related neurological disorder such as AD, VaD or DLB) may be indicated by a change in the level of clusterin detected over time when the results of two or more time intervals are compared for the same subject. In other words, if the method is performed a plurality of times, an improvement in disease status may be indicated when the level of clusterin detected at the later time interval(s) is different than that detected at the earlier time interval(s).

In one example, for determining the therapeutic effect of a treatment regimen for in a subject with AD specifically, an improvement in disease status or symptoms (e.g. over a treatment period) may be indicated by an increase in the level of clusterin detected over time when the results of two or more time intervals are compared for the same subject. In other words, if the method is performed a plurality of times, an improvement in disease status or symptoms (e.g. over a treatment period) may be indicated when the level of clusterin detected at the later time interval(s) is higher than that detected at the earlier time interval(s). An “increase” in the level of clusterin encompasses detection of clusterin at a later time interval when no clusterin was detected (i.e. it was not present at detectable levels) when the method was performed previously (i.e. at an earlier time interval) on the same subject (and an equivalent whole blood sample type).

Accordingly, in one example, step d) of the method described above for determining the therapeutic effect of a treatment regimen for dementia comprises identifying that the treatment regimen has a therapeutic effect if the level of clusterin in c) compared to b) is increased and the subject has Alzheimer's disease or is at increased risk of developing Alzheimer's disease.

Conversely, for determining the therapeutic effect of a treatment regimen for in a subject with VaD specifically, an improvement in disease status or symptoms (e.g. over a treatment period) may be indicated by a decrease in the level of clusterin detected over time when the results of two or more time intervals are compared for the same subject. In other words, if the method is performed a plurality of times, an improvement in disease status or symptoms (e.g. over a treatment period) may be indicated when the level of clusterin detected at the later time interval(s) is lower than that detected at the earlier time interval(s). An “decrease” in the level of clusterin encompasses no detection of clusterin (i.e. it is not present at detectable levels) at a later time interval when clusterin was detected when the method was performed previously (i.e. at an earlier time interval) on the same subject (and an equivalent whole blood sample type).

Accordingly, in one example, step d) of the method described above for determining the therapeutic effect of a treatment regimen for dementia comprises identifying that the treatment regimen has a therapeutic effect if the level of clusterin in c) compared to b) is decreased and the subject has Vascular dementia or is at increased risk of developing Vascular dementia.

Suitable time intervals for monitoring an improvement in disease status or symptoms (e.g. during treatment of the subject) can easily be identified by a person of skill in the art and will depend on the specific form of dementia (e.g. dementia symptom or dementia related neurological disorder e.g. AD, VaD or DLB) being monitored. As a non-limiting example, the method may be repeated at least every six months, or at least every year, or at least every two years, or more frequently as required (e.g. during treatment of the subject for dementia or a dementia related neurological disorder e.g. AD, VaD or DLB).

Methods for Determining a Subject's Compliance or Adherence with a Prescribed Treatment Regimen for Dementia

An in vitro method for determining a subject's compliance or adherence with a prescribed treatment regimen for dementia is also provided, the method comprising:

a) providing a whole blood sample from the subject;

b) determining the level of one or more biomarker in the whole blood sample, wherein the one or more biomarker is selected from the group consisting of clusterin, alpha-synuclein and amyloid precursor protein (APP);

c) repeating steps a) and b) after a time interval using a whole blood sample obtained from the subject after the prescribed start of treatment regimen; and

d) comparing the level of biomarker determined in step b) to that determined in step c), and identifying that the subject has complied or adhered with the prescribed treatment regimen if one or more of the following is observed: there is a change in the level of clusterin after treatment with the medicament; there is an increase in the level of alpha-synuclein after treatment; or there is an increase in the level of amyloid precursor protein after treatment.

Typically, the cause of the dementia is a dementia-related neurological disorder such as Alzheimer's disease (AD) or Vascular dementia (VaD) or Dementia with Lewy Bodies (DLB).

Appropriate subjects, treatments, terminology and permutations or combinations of features have been described in detail above.

The trends for identifying that the subject has complied or adhered with the prescribed treatment regimen are equivalent to those described in detail above in respect of determining the therapeutic effect of a treatment regimen for dementia. This is because a “prescribed treatment regimen” is a recommended treatment regimen and therefore typically has a therapeutic effect (and thus, observation of the therapeutic effect on the biomarker levels is an indication of subject compliance or adherence with the prescribed treatment regimen) Accordingly, all aspects described in detail above for methods for determining the therapeutic effect of a treatment regimen for dementia apply equally here.

Methods and Uses of the Biomarkers for Assessing Changes in Cognition/Cognitive Score

The biomarkers described herein may also be used as whole blood biomarkers for assessing cognition (e.g. equivalent to CAMCOG, MMSE or other suitable cognitive scores) in a subject having or at risk of having dementia. This is particularly the case when the cause of the dementia is a dementia-related neurological disorder such as Alzheimer's disease (AD), Vascular dementia (VaD) or Dementia with Lewy bodies (DLB).

An in vitro method for monitoring changes in cognition in a subject having or at risk of having dementia is therefore provided, the method comprising the steps of:

i) performing the following steps:
a) providing a whole blood sample from the subject;
b) determining the level of one or more biomarker in the whole blood sample, wherein the one or more biomarker is selected from the group consisting of clusterin, alpha synuclein (AS) and amyloid precursor protein (APP);
c) comparing the level of the one or more biomarker with the level of the same biomarker in a control sample or with a pre-determined reference level for the same biomarker to identify an increase or decrease in a level of the one or more biomarker in the sample of the subject;

ii) repeating i) for the same subject after a time interval;

iii) comparing the biomarker levels identified in i) with the biomarker levels identified in ii), and identifying a reduction in cognitive score if the comparison in step iii) indicates that the subject has one or more of the following: a change in the level of clusterin over the time interval; an increase in the level of alpha-synuclein over the time interval; or a decrease in the level of amyloid precursor protein over the time interval.

Appropriate subjects, treatments, terminology and permutations or combinations of features have been described in detail above.

Methods for determining cognitive scores (e.g. CAMCOG, MMSE etc) are described in detail elsewhere herein and are well known neurophysiological methods for assisting in dementia diagnosis. Advantageously, one or more of the biomarkers described herein [e.g. clusterin; AS; APP (e.g. APP-N); clusterin and AS; clusterin and APP; clusterin, AS and APP; AS and APP etc] can also be used to predict/monitor changes in cognitive score in a subject over time. Monitoring changes in cognitive score in a subject over time assists in the earliest possible identification of disease progression (e.g. a worsening in disease status or disease symptoms). Using the biomarkers described herein to monitor these changes is fast and reliable. This allows for early intervention and/or commencement of treatment at earlier stages of disease progression. It also provides a means for monitoring high risk individuals (i.e. individuals that are very likely or more likely to develop the disease) such that treatment or preventative measures may be put in place at the earliest opportunity. The methods described herein therefore can be used to detect, monitor and identify early stages of disease (or risk of disease).

Such monitoring naturally involves the taking of repeated samples over time. The method may therefore be repeated at one or more time intervals for a particular subject and the results compared to monitor for possible changes cognitive scores for that subject over time, wherein a reduction in cognitive score may be identified by a change in the level of clusterin over the time interval; an increase in the level of alpha-synuclein over the time interval; or a decrease in the level of amyloid precursor protein over the time interval.

Kits and Assay Devices for Assessing Changes in Cognition/Cognitive Score (e.g. CAMCOG and/or MMSE Scores, or Similar Cognitive Assessment Tests).

In another aspect, kits are provided for monitoring cognition in a subject having or at risk of having dementia. The kits include reagents suitable for determining levels of a plurality of analytes in a test sample (e.g., reagents suitable for determining levels of the biomarkers disclosed herein).

The kits described herein typically comprise:

(i) a detectably labelled agent that specifically binds to clusterin; and

(ii) one or more of:
c) a detectably labelled agent that specifically binds to alpha-synuclein; and
d) a detectably labelled agent that specifically binds to amyloid precursor protein (APP).

The kits may alternatively comprise a detectably labelled agent that specifically binds to alpha-synuclein and a detectably labelled agent that specifically binds to amyloid precursor protein (APP).

In some examples, the kit comprises a detectably labelled agent that specifically binds to alpha-synuclein, a detectably labelled agent that specifically binds to amyloid precursor protein (APP), and a detectably labelled agent that specifically binds to clusterin.

The kits described herein can take on a variety of forms. Typically, the kits will include reagents suitable for determining levels of a plurality of biomarkers (e.g., those disclosed herein, for example clusterin and AS, clusterin and APP, AS and APP, or clusterin, AS and APP) in a sample.

Optionally, the kits may contain one or more control samples or references. Typically, a comparison between the levels of the biomarkers in the subject and levels of the biomarkers in the control samples is indicative of a clinical status (e.g., a reduction or change in cognitive score (e.g. CAMCOG and/or MMSE etc score)). Also, the kits, in some cases, will include written information (indicia) providing a reference (e.g., pre-determined values), wherein a comparison between the levels of the biomarkers in the subject and the reference (pre-determined values) is indicative of a clinical status (e.g., a reduction or change in cognitive score (e.g. CAMCOG and/or MMSE etc score)). In some cases, the kits comprise software useful for comparing biomarker levels or occurrences with a reference (e.g., a prediction model). Usually the software will be provided in a computer readable format such as a compact disc, but it also may be available for downloading via the internet. However, the kits are not so limited and other variations with will apparent to one of ordinary skill in the art.

The components of the kit may be housed in a container that is suitable for transportation. Details on the biomarkers is given above and apply equally here. Suitably, the biomarker may be protein or mRNA.

The terms “detectably labelled agent”, “specifically binds to clusterin”, “specifically binds to AS”, “specifically binds to APP” etc are defined elsewhere herein and apply equally here.

In some examples the kits include the detectably labelled agent(s) on a continuous (e.g. solid) surface, such as a lateral flow surface. Alternatively, in examples comprising more than one detectably labelled agent, the detectably labelled agent(s) may be located in distinct (i.e. spatially separate) zones on a (e.g. solid) surface, such as a multiwall micro-titre plate (e.g. for an ELISA assay). Other appropriate surfaces and containers that are well known in the art may also form part of the kits described herein.

In one example, the kit further comprises one or more reagents for detecting the detectably labelled agent. Suitable reagents are well known in the art and include but are not limited to standard reagents and buffers required to perform any one of the appropriate detection methods that may be used (and are well known in the art). In one example, the kit comprises one or more of the following: a multi-well plate, ball bearing(s), extraction buffer, extraction bottle and a lateral flow device lateral flow device.

An assay device is also provided for monitoring cognition/cognitive scores in a subject having or at risk of having dementia.

Typically, the device comprises a surface with at least two detectably labelled agents located thereon, wherein the at least two detectably labelled agents are:

(i) a detectably labelled agent that specifically binds to clusterin; and

(ii) one or more of:
a detectably labelled agent that specifically binds to alpha-synuclein; and
a detectably labelled agent that specifically binds to amyloid precursor protein (APP).

The device may comprise a detectably labelled agent that specifically binds to clusterin, a detectably labelled agent that specifically binds to alpha-synuclein and a detectably labelled agent that specifically binds to amyloid precursor protein (APP).

The device may alternatively comprise a surface with at least two detectably labelled agents located thereon, wherein the at least two detectably labelled agents are:

(i) a detectably labelled agent that specifically binds to AS; and

(ii) one or more of:
a detectably labelled agent that specifically binds to clusterin; and
a detectably labelled agent that specifically binds to amyloid precursor protein (APP).

The device may alternatively comprise a surface with at least two detectably labelled agents located thereon, wherein the at least two detectably labelled agents are:

(i) a detectably labelled agent that specifically binds to APP; and

(ii) one or more of:
a detectably labelled agent that specifically binds to AS; and
a detectably labelled agent that specifically binds to amyloid precursor protein clusterin.

The at least two detectably labeled agents may be located in separate zones on the surface. In other words, the at least two detectably labelled agents may be located in distinct (i.e. spatially separate) zones on a (e.g. solid) surface, such as a multiwell micro-titre plate. Detectably labelled agent(s) that specifically bind to the biomarker(s) of interest are described in detail elsewhere herein.

The assay device comprises a surface upon which the detectably labelled agents are located. Appropriate surfaces include a continuous (e.g. solid) surface, such as a lateral flow surface, a dot blot surface, a dipstick surface or a surface suitable for performing surface plasmon resonance. Other appropriate surfaces include microtitre plates, multi-well plates etc. Other appropriate surfaces that are well known in the art may also form part of the assay device described herein.

Appropriate assay device formats therefore include but are not limited to device formats suitable for performing any one of lateral flow, dot blot, ELISA, or surface plasmon resonance assays for detecting the presence, level or absence of the biomarker of interest.

Data Storage Aspects

Biomarker levels and/or reference levels may be stored in a suitable data storage medium (e.g., a database) and are, thus, also available for future diagnoses. This also allows efficiently diagnosing prevalence for a disease because suitable reference results can be identified in the database once it has been confirmed (in the future) that the subject from which the corresponding reference sample was obtained did have dementia or a dementia related neurologic disorder. As used herein a “database” comprises data collected (e.g., analyte and/or reference level information and/or patient information) on a suitable storage medium. Moreover, the database, may further comprise a database management system. The database management system is, preferably, a network-based, hierarchical or object-oriented database management system. Furthermore, the database may be a federal or integrated database. More preferably, the database will be implemented as a distributed (federal) system, e.g. as a Client-Server-System. More preferably, the database is structured as to allow a search algorithm to compare a test data set with the data sets comprised by the data collection. Specifically, by using such an algorithm, the database can be searched for similar or identical data sets being indicative of dementia or a dementia related neurologic disorder (e.g. a query search). Thus, if an identical or similar data set can be identified in the data collection, the test data set will be associated with dementia or a dementia related neurologic disorder. Consequently, the information obtained from the data collection can be used to diagnose dementia or a dementia related neurologic disorder or based on a test data set obtained from a subject. More preferably, the data collection comprises characteristic values of all analytes comprised by any one of the groups recited above.

The methods described herein may further include communication of the results or diagnoses (or both) to technicians, physicians or patients, for example. In certain examples, computers will be used to communicate results or diagnoses (or both) to interested parties, e.g., physicians and their patients.

In some examples, the results or diagnoses (or both) are communicated to the subject as soon as possible after the diagnosis is obtained. The results or diagnoses (or both) may be communicated to the subject by the subject's treating physician. Alternatively, the results or diagnoses (or both) may be sent to a subject by email or communicated to the subject by phone. A computer may be used to communicate the results or diagnoses by email or phone. In certain examples, the message containing results or diagnoses may be generated and delivered automatically to the subject using a combination of computer hardware and software which will be familiar to artisans skilled in telecommunications.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1 94); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Aspects of the invention are demonstrated by the following non-limiting examples.

### Examples

Methods:

Patient Data

A study was conducted on whole blood samples obtained from a total number of 123 subjects, this included: control subjects (N=48), AD (N=33), VaD (N=24) and DLB (N=18) patients. Demographic (gender and age) and extensive clinical data for all subjects enrolled in the study was obtained. Thus, both control, and dementia subjects underwent cognitive assessment using the CAMCOG (Cambridge Cognitive Examination) and MMSE (Mini Mental State Examination), whereas dementia patients had additional assessments for their behaviour, which included NPI (Neuropsychiatric Inventory) and Cornell (Depression Scale) (Mukaetova-Ladinska et al., 2012). It is noted that the inventors did not have access to DLB drug-naive patients, therefore data for DLB drug-naive patients is not included in the table of data below.

Control Data

The control data were generated from samples of whole blood that came from subjects who did not have memory problems, and no diagnosis of dementia. They also were devoid of any neurological and physical conditions that may underlie dementia. The control samples came from healthy individuals, usually spouses of the dementia patients enrolled in the study and volunteers. All control subjects had detailed clinical history taken, medication history, and underwent cognitive testing, consisting of CAMCOG (that also includes MMSE) on the day they donated their blood samples.

Blood Samples

Whole blood samples were obtained from the patients following the clinical assessments. As per standard practice, the blood was withdrawn from a patient into an anticoagulant solution (e.g. EDTA) and transported to the lab. Once the blood samples were received in the lab, 1 ml of whole blood was reserved and frozen at −40° C. Once defrosted the blood sample was mixed (vortexed) and used directly in the ELISA assays described herein. Please note that no modifications were used (i.e. the whole blood sample that was tested in the ELISA assay comprised all of the components of blood (i.e. white and red blood cells, platelets, and plasma).

Indirect ELISA Immunoassay

Indirect enzyme linked immunoabsorbent assays (ELISAs) were used to determine protein levels of each of the biomarkers in whole blood samples (using equivalent methods to those described in Mukaetova-Ladinska et al, 2012). As an example, a brief protocol is provided for the detection of the AS biomarker. This protocol applies equally to the other biomarkers described herein, using commercially available antibodies/detection kits.

TABLE 1
The primary immunoprobes, and secondary antibodies used in ELISA
immunoassays.

| | Primary Antibody | Secondary |
|---|---|---|
| Primary Antibody | Dilution in PBS | Antibody Dilution |
| (Company) | Tween | (Company) |
| Polyclonal α- | 1:5000 | Polyclonal rabbit |
| synuclein (Santa | | HRP 1:1000 (Dako) |
| Cruz Biotech) | | |
| Polyclonal APP-N | 1:1000 | Polyclonal rabbit |
| (Sigma) | | HRP 1:1000 (Dako) |
| Monoclonal clusterin | 1:1000 | Polyclonal mouse |
| (Santa Cruz | | HRP 1:1000 (Dako) |
| Biotech) | | |

For the detection of AS, whole blood samples were loaded to the wells of a 96 plate at a 1:20 dilution (diluted in coating buffer 50 mM concentration of carbonate-bicarbonate buffer and a pH level of 9.6). The plates were then incubated overnight at 4° C. The following day they were washed using 0.05% Tween and wells were blocked with 1% Marvel in phosphate buffer solution (PBS pH 6.8) and incubated for an hour at 37° C. Plates were washed again and coated with a primary antibody against the C-terminal end of AS (pAb α-synuclein [(C-20)-R; SANTA CRUZ Biotechnology, INC]), and diluted at 1:5000 with 1% Tween in PBS. The plates were then incubated for 1 hour at 37° C. and washed again. Following this, all wells were coated with a secondary antibody conjugated to horseradish peroxide (HRP) (anti-rabbit-HRP P0448, DAKO Cytomation, Gostrup, Denmark) and incubated for 1 hour at 37° C. Fresh substrate reagent [3,3′, 5, 5′-trimethylbenzidine (TMB; Sigma), sodium acetate and hydrogen peroxide] was then added to the wells to perform a colorimetric analysis and the reaction was sequentially quenched after 10 minutes using H₂SO₄. The plates were read with a Vmax platereader to produce the data.

Statistical Analysis

The data were analysed using SPSS v.24. Normality of data was established with the Kolmogorov-Smirnov Test. Since data were non-parametrically distributed non-parametric analysis was used to establish the differences between dementia and control subject with the Kruskal-Wallis test. Sensitivity and specificity of protein concentration and biomarker measures was established with Receiver Operating Characteristics (ROC) curve. Spearman correlation analysis was used to determine the relationship between the different ELISA data, whereas the relationship between clinical measures of cognitive and behavioural changes and biochemical measures with regression analysis. Statistical significance was set at p=≤0.05.

Results:

The following table summarises the data that was generated by the inventors:

TABLE 3
summary of data showing clusterin, alpha synuclein and APP as biomarkers for dementia.
The number in brackets in the farthest left column, represent the number of cases from
which the mean values are derived.

| Measures | Controls | AD | VaD | DLB | ANOVA | P value |
|---|---|---|---|---|---|---|
| Age/years | 73.48 ± 1.59 | 80.12 ± 1.06 | 79.92 ± 1.20 | 77.89 ± 1.44 | 5.354 | 0.002 |
| (50/34/25/18) | | | | | | |
| CAM COG | 94.02 ± 0.73 | 72.18 ± 2.16 | 61.88 ± 4.12 | 68.94 ± 5.02 | 37.739 | 0.0001 |
| (50/34/25/17) | | | | | | |
| MMSE | 27.67 ± 0.28 | 21.12 ± 0.77 | 18.60 ± 1.21 | 19.44 ± 1.73 | 29.218 | 0.0001 |
| (49/34/25/18) | | | | | | |
| Cornell | NA | 5.82 ± 0.81 | 6.09 ± 0.70 | 7.11 ± 1.34 | 0.468 | 0.628 |
| (0/34/23/18) | | | | | | |
| NPI | NA | 11.77 ± 1.87 | 10.65 ± 1.71 | 16.33 ± 3.96 | 1.274 | 0.286 |
| (0/34/23/28) | | | | | | |
| Fluctuation | NA | 1.94 ± 0.32 | 2.39 ± 0.59 | 3.11 ± 0.89 | 1.086 | 0.343 |
| (0/34/23/28) | | | | | | |
| Clusterin | 125.41 ± 2.76 | 104.42 ± 3.74 | 159.09 ± 10.06 | | 26.703 | 0.0001 |
| Drug naïve | | | | | | |
| (48/33/11) | | | | | | |
| 1 m (33) | | 135.30 ± 32.44 | | | | |
| 6 m | | 115.04 ± 5.85 | 135.19 ± 12.22 | 144.45 ± 130.26 | 4.707 | 0.013 |
| (27/12/16) | | | | | | |
| Alpha | 246.43 ± 28.28 | 358.76 ± 37.87 | 375.85 ± 61.44 | | 3.267 | 0.031 |
| synuclein | | | | | | |
| Drug naïve | | | | | | |
| (48/33/8) | | | | | | |
| 1 m (20) | | 294.81 ± 50.34 | | | | |
| 6 m (9/11/18) | | 574.31 ± 53.54 | 517.77 ± 75.93 | 423.58 ± 61.96 | 1.315 | 0.281 |
| APP | 110.83 +± 5.27 | 91.93 ± 4.05 | 84.17 ± 10.70 | | 5.050 | 0.008 |
| Drug naïve | | | | | | |
| (45/32/10) | | | | | | |
| 1 m (22) | | 95.27 ± 4.74 | | | | |
| 6 m (8/11/14) | | 104.17 ± 7.22 | 103.22 ± 8.73 | 136.53 ± 9.50 | 4.726 | 0.016 |

In table 3, ANOVA values are listed. The ANOVA values were obtained using the SPSS v.24.0. Significance of analysis was set at p=0.05, to provide 95% confidence interval. All results that had a p value <0.05 were statistically significant. The lower the value, the more significant the findings/differences were.

Whole blood samples can be used to compare the level of the one or more biomarker described herein with the level of the same biomarker in a control sample or with a pre-determined reference level for the same biomarker. An increase or decrease in the level of the one or more biomarker in the sample of the subject compared to the control/reference level value can be used to identify a subject as having dementia or as having an increased risk of developing dementia. This is particularly the case when the comparison indicates one or more of the following: a change in the level of clusterin compared to the control sample or the pre-determined reference level; a decreased level of amyloid precursor protein compared to the control sample or the pre-determined reference level; or an increased level of alpha-synuclein compared to the control sample or the pre-determined reference level.

Clusterin can be used as a biomarker in whole blood samples for dementia, for example as a biomarker for Alzheimer's disease (AD), Vascular dementia (VaD) or Dementia with Lewy Bodies (DLB). The data herein indicate that it is possible to use clusterin as a biomarker in whole blood for Alzheimer's disease as a subject with a decreased level of clusterin compared to a control sample or a pre-determined reference level may be identified as having Alzheimer's disease or as having an increased risk of developing Alzheimer's disease. Conversely, the data indicate that it is possible to use clusterin as a biomarker in whole blood for Vascular dementia as a subject with an increased level of clusterin compared to a control sample or a pre-determined reference level may be identified as having Vascular dementia or as having an increased risk of developing Vascular dementia.

The data also indicate that alpha synuclein (AS) can be used as a biomarker in whole blood samples for dementia, for example as a biomarker for Alzheimer's disease (AD), Vascular dementia (VaD) or Dementia with Lewy Bodies (DLB). The data herein indicate that it is possible to use AS as a biomarker in whole blood for Alzheimer's disease or Vascular dementia as a subject with an increased level of AS compared to a control sample or a pre-determined reference level may be identified as having Vascular dementia or Alzheimer's disease or as having an increased risk of developing Vascular dementia or Alzheimer's disease.

The data also indicate that amyloid pre-cursor protein (APP) can be used as a biomarker in whole blood samples for dementia, for example as a biomarker for Alzheimer's disease (AD), Vascular dementia (VaD) or Dementia with Lewy Bodies (DLB). The data herein indicate that it is possible to use APP as a biomarker in whole blood for Alzheimer's disease or Vascular dementia as a subject with a decreased level of APP compared to a control sample or a pre-determined reference level may be identified as having Vascular dementia or Alzheimer's disease or as having an increased risk of developing Vascular dementia or Alzheimer's disease.

Advantageously one or more (e.g. two or three) of these biomarkers can be used in combination as combined biomarkers in whole blood samples for dementia, such as Alzheimer's disease (AD), Vascular dementia (VaD) or Dementia with Lewy Bodies (DLB). The trends observed for the biomarkers singularly can therefore be combined to give a more accurate and/or sensitive predictive score.

The data generated herein demonstrate for the first time that whole blood samples can be used as biomarkers for diagnosing dementia or the risk of developing dementia, and can be used to distinguish between AD, VaD and DLB. These biomarkers are therefore also useful for monitoring dementia progression over time.

Measurement of these biomarkers may also be useful when determining the therapeutic effect of appropriate treatment regimens or determining a subject's compliance or adherence with a prescribed treatment regimen. This is supported by the data shown in Table 3 for drug naïve patients vs patients that have been treated for one month or six months.

Patients that were used to obtain values for biomarker levels after 1 month of treatment and after 6 months of treatment were all on standard accepted treatment regimens for the indicated form of dementia. A table summarising the prescribed medication for these patients is shown below:

TABLE 4
Summary of antidementia drugs participants were treated with.

| Antidementia treatment | AD | VaD | DLB |
|---|---|---|---|
| Donepezil | 33 | 7 | 12 |
| Rivastigmine | | | 4 |
| Galantamine | | | |
| Memantine | | 5 | |
| Donepezil + memantine | | | 1 |
| Rivastigmine + memantine | | | |
| Galantamine + memantine | | | 1 |

As shown in Table 3, treatment of dementia (e.g. AD or VaD) using standard accepted forms of medication for these disorders affects the level of clusterin in whole blood over the treatment period tested, such that it more closely resembles control values when compared to the level of clusterin in whole blood samples taken from corresponding drug naïve demented patients. By way of example, treatment reduces the clusterin level in VaD patient whole blood over time, bringing it more in line with control levels. Conversely, treatment increases the clusterin level in AD patient whole blood over time, bringing it more in line with control levels. By extrapolation, although drug naïve DLB patient data was not available, it would be expected that treatment of DLB patients is likely to reduce clusterin levels in whole blood over time to bring it more in line with control levels (i.e. that in drug naïve DLB patients, levels of clusterin are elevated compared to controls, and are reduced to be more in line with control during treatment (see elevated levels of clusterin observed in DLB patients that have been treated for 6 months)).

As another example, as shown in Table 3, treatment of dementia (e.g. AD or VaD) using standard accepted forms of medication for these disorders affects the level of AS in whole blood, specifically, increases the level of AS in whole blood over the treatment period tested. By extrapolation, although drug naïve DLB patient data was not available, it would be expected that treatment of DLB patients is likely to increase AS levels in whole blood over time (i.e. that in drug naïve DLB patients, levels of AS are elevated compared to controls, and then increase further during treatment (see elevated levels of AS observed in DLB patients that have been treated for 6 months)).

As another example, as shown in Table 3, treatment of dementia (e.g. AD or VaD) using standard accepted forms of medication for these disorders affects the level of APP (e.g. APP-N) in whole blood over the treatment period tested, such that it more closely resembles control values compared to the level of APP (e.g. APP-N) in whole blood samples taken from corresponding drug naïve demented patients. By way of example, treatment increases the APP level in AD and VaD patient whole blood over time, bringing it more in line with control levels. based on the DL pilot data presented here the DLB treated subjects have higher levels of APP in relation to the control and AD and VaD treated participants (p=0.016). One of the explanations for this may be due to the blood APP holoprotein being modulated by iron by a mechanism similar to the translation control of ferritin by iron-responsive element, that has been also hypothesised to exist in alpha-synucleinopathies, including DLB and Parkinson's disease (Friedlich et al., 2007).

The CAMCOG and MMSE data in Table 3 also show that the specified biomarkers (i.e. clusterin, AS and APP) can be used as biomarkers to identify a change, a risk of change, or predict a change in CAMCOG or MMSE score for a patient having or at risk of having dementia (e.g. AD, VaD or DLB).

The data shows that patients with AD, AS and DLB all have lower CAMCOG and/or MMSE scores compared to controls. These data indicate that the correlations described above with reference to identifying dementia or the risk thereof equally apply to identifying a decrease in (or a risk of a decrease in) CAMCOG and/or MMSE score. Clusterin, AS and/or APP can therefore be used as useful biomarkers to identify a change, a risk of change, or predict a change in CAMCOG or MMSE score, or alternative cognitive assessment tool score for a patient having or at risk of having dementia (e.g. AD, VaD or DLB).

In other words, in a patient that has or is at risk of having dementia (e.g. AD, VaD or DLB), one or more of: a change in the level of clusterin compared to a control sample or a pre-determined reference level; a decreased level of amyloid precursor protein compared to a control sample or a pre-determined reference level; and/or an increased level of alpha-synuclein compared to a control sample or a pre-determined reference level; may be used as an indication of a decrease (or risk of a decrease, or a predicted decrease) in CAMCOG or MMSE score, or an alternative cognitive score for that patient.

As an example, clusterin can be used as a biomarker in whole blood samples as an indication of a change in, a risk of change, or to predict a change in CAMCOG and/or MMSE score, or an alternative cognitive score, for a patient having or at risk of having dementia (e.g. AD, VaD or DLB). The data herein indicate that it is possible to use clusterin as a biomarker in whole blood as an indicator of changes in cognition, as assessed with CAMCOG and/or MMSE score, or an alternative cognitive score, as an AD subject with a decreased level of clusterin compared to a control sample or a pre-determined reference level may be identified as having a reduced cognitive (i.e. CAMCOG and/or MMSE score, or similar) compared to control or as having an increased risk of developing a reduced CAMCOG and/or MMSE score (or similar) compared to control. Conversely, the data indicate that it is possible to use clusterin as a biomarker in whole blood as an indicator of changes in CAMCOG and/or MMSE score, or similar, as a VaD subject with an increased level of clusterin compared to a control sample or a pre-determined reference level may be identified as having a reduced CAMCOG and/or MMSE score, or similar cognitive assessment score, compared to control or as having an increased risk of developing a reduced CAMCOG and/or MMSE score, or alternative, compared to control. This is particularly useful when monitoring the progression of dementia over time, or monitoring the risk of developing dementia over time, or monitoring CAMCOG and/or MMSE scores (or alternative) over time for one particular individual, as a clusterin value at the start (or at earlier time points) of the monitoring process can be used as the control for comparison with later time point clusterin values (i.e. to identify if there is a change in AS level over time (as an indication of a change or risk of change, or a prediction of change, in CAMCOG and/or MMSE score, or alternative cognitive score, over time for that particular individual)).

The data also indicate that alpha synuclein (AS) can be used as a biomarker in whole blood samples as an indication of a change in, a risk of change, or to predict a change in CAMCOG and/or MMSE score, or alternative cognitive score, for a patient having or at risk of having dementia (e.g. AD, VaD or DLB). The data herein indicate that it is possible to use AS as a biomarker in whole blood as an indicator of changes in CAMCOG and/or MMSE score, or alternative cognitive score, as a subject with an increased level of AS compared to a control sample or a pre-determined reference level may be identified as having a reduced CAMCOG and/or MMSE score, or alternative cognitive score, compared to control or as having an increased risk of developing a reduced CAMCOG and/or MMSE score (or alternative cognitive score) compared to control. This is particularly useful when monitoring the progression of dementia over time, or monitoring the risk of developing dementia over time, or monitoring CAMCOG and/or MMSE scores (or alternative cognitive score) over time for one particular individual, as an AS value at the start (or at earlier time points) of the monitoring process can be used as the control for comparison with later time point AS values (i.e. to identify if there is a change in AS level over time (as an indication of a change or risk of change, or a prediction of change, in CAMCOG and/or MMSE score (or alternative cognitive score) over time for that particular individual)).

The data also indicate that amyloid precursor protein (APP) can be used as a biomarker in whole blood samples as an indication of a change in, a risk of change, or to predict a change in CAMCOG and/or MMSE score, or alternative cognitive score, for a patient having or at risk of having dementia (e.g. AD, VaD or DLB). The data herein indicate that it is possible to use APP as a biomarker in whole blood as an indicator of changes in CAMCOG and/or MMSE score, or alternative cognitive score, as a subject with a decreased level of APP compared to a control sample or a pre-determined reference level may be identified as having a reduced CAMCOG and/or MMSE score (or alternative cognitive score) compared to control or as having an increased risk of developing a reduced CAMCOG and/or MMSE score (or alternative cognitive score) compared to control. This is particularly useful when monitoring the progression of dementia over time for an individual, or monitoring the risk of developing dementia over time for an individual, or monitoring CAMCOG and/or MMSE scores, or alternative cognitive score, over time for an individual, as an APP value at the start (or at earlier time points) of the monitoring process can be used as the control for comparison with later time point APP values (i.e. to identify if there is a change in APP level over time (as an indication of a change or risk of change, or a prediction of change, in CAMCOG and/or MMSE score, or alternative cognitive score, over time for that particular individual)).

Advantageously one or more (e.g. two or three) of these biomarkers can be used in combination as combined biomarkers in whole blood samples as an indication of a change in CAMCOG and/or MMSE score, or alternative cognitive score, for a patient having or being at risk of having dementia (e.g. AD, VaD or DLB). The trends observed for the biomarkers singularly can therefore be combined to give a more accurate and/or sensitive predictive score or indication of CAMCOG and/or MMSE score, or alternative cognitive score, or a change in CAMCOG and/or MMSE score, or alternative cognitive score, over time.

The data generated herein demonstrate for the first time that whole blood samples can be used to measure clusterin, AS and APP and that the levels of clusterin, AS and APP in whole blood can be used as biomarkers for a change in CAMCOG and/or MMSE score. These biomarkers are therefore useful for monitoring a change in CAMCOG and/or MMSE score over time. Since the most widely used cognitive assessment tool, ACE-IIII is highly significantly correlated with MMSE (Velayudhan et al, 2014), which is contained also within the CAMCOG, the above statements of our whole blood biomarkers be used for monitoring a change in cognition, as measured with CAMCOG and MMSE, can be extended to other cognitive assessment tools, such as ACE-Ill, and similar.

### REFERENCES

Luzny, J., Holmerova, I., Wija, P. & Ondrejka, I. (2014) Dementia Still Diagnosed Too Late—Data from the Czech Republic. Iranian Journal of Public Heath, 43(10) pp. 1436-1443.
Bunn, F., Burn, A. M., Goodman, C., Rait, G., Norton, S., Robinson, L., Schoeman, J., & Brayne, C., (2014) ‘Comorbidity and dementia: a scoping review of the literature’ BMC Medicine. 12(192)
Rizzi, L., Rosset, I., & Roriz-Cruz, M., (2014) ‘Global Epidemiology of Dementia: Alzheimer's and Vascular Types’ BioMed Research International. [Online]. Available at: doi:10.1155/2014/908915. (Accessed: 4 Jun. 2015)
Wurtman, R., (2015) ‘Biomarkers in the diagnosis and management of Alzheimer's disease’ Metabolism. 64(3): S47-S50
Chiam, J. T. W., Dobson, R. J. B., Kiddle, S. J., & Sattlecker, M., (2015) ‘Are BloodBased Protein Biomarkers for Alzheimer's Disease also Involved in Other Brain Disorders? A Systematic Review’ Journal of Alzheimer's Disease. 43: 303-314
Kumar, A., Singh, A. & Ekavali. (2014) A review on Alzheimer's disease pathophysiology and its management: an update. Pharmalogical Reports, 67(2), pp. 195-203.
Alzheimer's Association (2011) ‘2011 Alzheimer's disease facts and figures’ Alzheimer's Association Report. 7(2): 208-44
Rembach, A., Hare, J., D., Doecke, J., D., Burnham, S., C., Volitakis, I., Fowler, J., C., Cherny, R., A., McLean, C., Grimm, R., Martins, R., Ames, D., Masters, C., L., Busha, A., I. & Roberts, B., R. (2014) Decreased serum zinc is an effect of ageing and not Alzheimer's disease. Metabollomics, 6, pp. 1216-1219
Foster, V., Oakley, A., E., Slade, J., Y., Hall, R., Polvikoski, T., M., Burke, M., Thomas, A., J., Khundakar, A., Allan, L., M. & Kalaria, R., N. (2014) Pyramidal neurons of the prefrontal cortex in post-stroke, vascular and other ageing-related dementias. A Journal of Neurology. [Online] Available at: http://dx.doi.org/10.1093/brain/awu172. (Accessed on Jun. 5, 2015)
Khan, T. K., & Alkon, D., (2015) ‘Peripheral biomarkers of Alzheimer's disease.’ Journal of Alzheimer's disease. 44(3)
Maki, Y. & Yamaguchi, H. (2014) Early Detection of dementia in the community uner a community-based integrated care system. Geriatrics & Gerontology International, 14(2), pp. 2-10
Masellis, M., Sherborn, K., Neto, P., N., Sadovnick, D., A., Hsiung, G-Y., R., Black, S., E., Prasad, S., Williams, M. & Gauthier, S. (2013) Early-onset dementias: diagnostic and etiological considerations. Alzheimer's Research & Therapy, 5(1)
Hall, D. & Finger, E., C. (2015) Psychotic symptoms in frontotemporal dementia. Current Neurology and Neuroscience Reports, 15(7), pp. 567
Boot, B., P. (2015) Comprehensive treatment of dementia with Lewy bodies. Alzheimer's Research & Therapy, 7(1) pp. 45
Mukaetova-Ladinska, E., B. (2015) Molecular imaging biomarkers for dementia with Lewy bodies: an update. International Psychogeriatrics, 27(4), pp. 555-577
Gupta, S., Fiertag, O., Thanulingam, T., Ros, E., Strange, B. & Warner, J. (2012) Further rare and unusual dementias. Advances in Psychiatric Treatment, 18(1), pp. 67-77.
Cooper, S. & Greene, J., D., W. (2005) The clinical assessment of the patient with early dementia. The Journal of Neurology, Neurosurgery and Psychiatry, 76(5), pp. 15-24
Tsolaki, M. (2014) Clinical workout for the early detection of cognitive decline and dementia. European Journal of Clinical Nutrition, 68, pp. 1186-1191
Mukaetova-Ladinska, E., Abdel-All, Z., Dodds, S., Andrade, J., Alves da Silva, J., Kalaria, R. and O'Brien, J. (2012). Platelet immunoglobulin and amyloid precursor protein as potential peripheral biomarkers for Alzheimer's disease: findings from a pilot study. Age and Ageing, 41(3), pp. 408-412
Skillback, T., Farahmand, B., Y., Rosen, C., Mattsson, N., Nagga, Katarina., Kilander, L., Religa, D., Wimo, A., Winblad, B., Schott, J., M., Blennow, K., Fridcdotter, M. & Zetterberg, H. (2015) Cerebrospinal fluid tau and amyloid-β1-42 in patients with dementia. A Journal of Neurology, [Online] Availible at: http://dx.doi.org/10.1093/brain/awv181. (Accessed on 5 Jul. 2015)
Sweeney, M., D., Sagare, A., P. & Zlokovic, B., V. (2015) Cerebrospinal fluid biomarkers of neurovascular dysfunction in mild dementia and Alzheimer's disease. Journal of Cerebral Blood Flow & Metabolism, 35, pp. 1055-1068
De Sole, P., Rossi, C., Chiarpotto, M., Ciasca, G., Bocca, B., Alimonti, A., Bizzarro, A., Rossi, C. & Masullo, C. (2013) Possible relationship between Al/ferritin complex and Alzheimer's disease. Clinical Biochemistry, 46(1-2), pp. 89-93
Snyder, H., M., Carrillo, M., C., Grodstein, F., Henriksen, K., Jeromin, A., Lovestone, S., Mielke, M., M., O'Bryant, S., Sarasa, M., Siogren, M., Soares, H., Teeling, J., Trushina, E., Ward, M., West, T., Bain, L., J., Shineman, D., W., Weiner, M. & Fillit, H., M. (2014) Developing novel blood-based biomarkers for Alzheimer's disease. Alzheimer's & Dementia: the journal of the Alzheimer's Association, 10(1), pp. 109-114
Michell, A., W., Luheshi, L., M. & Barker, R., A. (2005) Skin and platelet alpha-synuclein as peripheral biomarkers of Parkinson's disease. Neuroscience Letters, 381(2), pp. 294-298
Schmidt M et al., Proc Natl Acad Sci USA. 2015 Sep 22; 112(38):11858-63. Peptide dimer structure in an Aβ(1-42) fibril visualized with cryo-EM.
Desai et al., Neurology. 2005 Jun. 28; 64(12 Suppl 3):534-9.Diagnosis and treatment of Alzheimer's disease.
McKhann et al. The diagnosis of dementia due to Alzheimer's disease: recommendations from the National Institute on Aging-Alzheimer's Association workgroups on diagnostic guidelines for Alzheimer's disease. Alzheimers Dement. 2011; 7: 263-269
Dubois et al. Advancing research diagnostic criteria for Alzheimer's disease: the IWG-2 criteria. Lancet Neurol. 2014; 13: 614-629.
McKeith et al., Neurology. 2017 Jul. 4; 89(1):88-100. Diagnosis and management of dementia with Lewy bodies: Fourth consensus report of the DLB Consortium.
Mukaetova-Ladinska E B, Abdel-All Z, Mugica E S, Li M, Craggs L J, Oakley A E, Honer W G, Kalaria R N. Tau proteins in the temporal and frontal cortices in patients with vascular dementia. J Neuropathol Exp Neurol. 2015 February; 74(2):148-57.
Kim W S, Kågedal K, Halliday GM. Alpha-synuclein biology in Lewy body diseases. Alzheimers Res Ther. 2014 Oct. 27; 6(5):73.
Sachdev P, Kalaria R, O'Brien J, Skoog I, Alladi S, SE, Blacker D, Blazer D, Chen C, Chui H, Ganguli M, Jellinger K, Jeste D V, Pasquier F, Paulsen J, Prins N, Rockwood K, Roman G, Scheltens P. Diagnostic criteria for vascular cognitive disorders: a VASCOG statement. Alzheimer Dis Assoc Disord. 2014 July-September; 28(3): 206-218.

A. L. Friedlich, R. E. Tanzi, J. T. Rogers, The 5′-untranslated region of Parkinson's disease alpha-synuclein messenger RNA contains a predicted iron responsive element, Mol. Psychiatry 12 (2007) 222-223.

Velayudhan L, Ryu S H, Raczek M, Philpot M, Lindesay J, Critchfield M, Livingston G. Review of brief cognitive tests for patients with suspected dementia. Int Psychogeriatr. 2014 Aug; 26(8):1247-62.

1. An in vitro method for diagnosing dementia or determining the risk of developing dementia in a subject, the method comprising the steps of:
a) providing a whole blood sample from the subject;
b) determining the level of one or more biomarker in the whole blood sample, wherein the one or more biomarker is selected from the group consisting of clusterin, alpha synuclein (AS) and amyloid precursor protein (APP);
c) comparing the level of the one or more biomarker with the level of the same biomarker in a control sample or with a pre-determined reference level for the same biomarker to identify an increase or decrease in a level of the one or more biomarker in the sample of the subject compared to the control sample or pre-determined reference value; and
d) identifying a subject as having dementia or as having an increased risk of developing dementia if the comparison in step c) indicates that the subject has one or more of the following: a change in the level of clusterin compared to the control sample or the pre-determined reference level; an increased level of alpha-synuclein compared to the control sample or the pre-determined reference level; or a decreased level of amyloid precursor protein compared to the control sample or the pre-determined reference level.

2. The method of claim 1, wherein step b) comprises the determining the level of at least two biomarkers selected from the group consisting of clusterin, alpha-synuclein and amyloid precursor protein.

3. The method of claim 1, wherein the cause of dementia is a dementia-related neurological disorder, such as Alzheimer's disease (AD), Vascular dementia (VaD) or Dementia with Lewy Bodies.

4. The method of claim 1, comprising identifying a subject with a decreased level of clusterin compared to the control sample or the pre-determined reference level as having Alzheimer's disease or as having increased risk of developing Alzheimer's disease.

5. The method of claim 1, comprising identifying a subject with an increased level of clusterin compared to the control sample or the pre-determined reference level as having Vascular dementia or as having increased risk of developing Vascular dementia.

6. The method of claim 1, wherein the level of biomarker is determined at the protein level, optionally using a process selected from the group consisting of immunoblotting, lateral flow assay, ELISA assay, protein microarray and mass spectrometry.

7. The method of claim 1, wherein the control sample is obtained from a non-demented control subject.

9. The method of claim 1, wherein the pre-determined reference level is the average level of the biomarker in a non-demented control subject.

10. The method of claim 1, wherein the subject is a human.

11. The method of claim 1, further comprising selecting a treatment for the subject based on the comparison of the level of the biomarker with the control sample or with the pre-determined reference level.

12. The method of claim 11, further comprising administering the selected treatment to the subject, optionally wherein the selected treatment comprises an effective amount of at least one anti-dementia compound.

13. The method of claim 12, wherein the anti-dementia compound is:
a) a cholinesterase inhibitor, optionally wherein the cholinesterase inhibitor is selected from the group consisting of donepezil, rivastigmine, galantamine, tacrine, or salts thereof, and/or b) an NMDA antagonist, optionally wherein the NMDA antagonist is memantine.

14. A kit for diagnosing dementia or determining the risk of developing dementia in a subject, comprising:
(i) a detectably labelled agent that specifically binds to clusterin; and
(ii) one or more of:
a) a detectably labelled agent that specifically binds to alpha-synuclein; and
b) a detectably labelled agent that specifically binds to amyloid precursor protein (APP).

15. The kit of claim 14, wherein the kit comprises a) and b).

16. The kit of claim 14, further comprising one or more reagents for detecting the detectably labelled agent(s).

17. An assay device for diagnosing dementia or determining the risk of developing dementia in a subject, the device comprising a surface with at least two detectably labelled agents located thereon, wherein the at least two detectably labelled agents are:
(i) a detectably labelled agent that specifically binds to clusterin; and
(ii) one or more of:
a) a detectably labelled agent that specifically binds to alpha-synuclein; and
b) a detectably labelled agent that specifically binds to amyloid precursor protein (APP).

18. The assay device of claim 17, wherein the device comprises a) and b).

19. The assay device according to claim 17, wherein the at least two detectably labeled agents are located in separate zones on the surface.

20. Use of one or more biomarkers selected from the group consisting of clusterin, alpha-synuclein and amyloid precursor protein (APP) as a whole blood biomarker for dementia.

21. The use according to claim 20, wherein the cause of the dementia is a dementia-related neurological disorder, such as Alzheimer's disease (AD), Vascular dementia (VaD) or Dementia with Lewy Bodies (DLB).

22. An in vitro method for monitoring dementia progression in a subject, the method comprising the steps of:
i) determining the level of one or more biomarker in a whole blood sample from the subject in accordance with method steps a) to c) of claim 1; and
ii) repeating step i) for the same subject after a time interval; and
iii) comparing the biomarker levels identified in i) with the biomarker levels identified in ii), wherein a change in the biomarker levels from i) to ii) is indicative of a change in dementia progression in the subject.

23. An in vitro method for determining the therapeutic effect of a treatment regimen for dementia, the method comprising:
a) providing a whole blood sample from the subject;
b) determining the level of one or more biomarker in the whole blood sample, wherein the one or more biomarker is selected from the group consisting of clusterin, alpha-synuclein and amyloid precursor protein (APP);
c) repeating steps a) and b) using a whole blood sample obtained from the subject after treatment for a time interval; and
d) comparing the level of biomarker determined in step b) to that determined in step c), and identifying that the treatment regimen has a therapeutic effect if one or more of the following is observed: there is a change in the level of clusterin after treatment; there is an increase in the level of alpha-synuclein after treatment; or there is an increase in the level of amyloid precursor protein after treatment.

24. The method of claim 22, wherein the cause of the dementia is a dementia-related neurological disorder such as Alzheimer's disease (AD) or Vascular dementia (VaD).

25. The method of claim 23, wherein step d) comprises identifying that the treatment regimen has a therapeutic effect if the level of clusterin in c) compared to b) is increased and the subject has Alzheimer's disease or is at increased risk of developing Alzheimer's disease.

26. The method of claim 23, wherein step d) comprises identifying that the treatment regimen has a therapeutic effect if the level of clusterin in c) compared to b) is decreased and the subject has Vascular dementia or is at increased risk of developing Vascular dementia.

27. An in vitro method for determining a subject's compliance or adherence with a prescribed treatment regimen for dementia, the method comprising:
a) providing a whole blood sample from the subject;
b) determining the level of one or more biomarker in the whole blood sample, wherein the one or more biomarker is selected from the group consisting of clusterin, alpha-synuclein and amyloid precursor protein (APP);
c) repeating steps a) and b) after a time interval using a whole blood sample obtained from the subject after the prescribed start of treatment regimen; and
d) comparing the level of biomarker determined in step b) to that determined in step c), and identifying that the subject has complied or adhered with the prescribed treatment regimen if one or more of the following is observed: there is a change in the level of clusterin after treatment with the medicament; there is an increase in the level of alpha-synuclein after treatment; or there is an increase in the level of amyloid precursor protein after treatment.

28. The method of claim 27, wherein the cause of the dementia is a dementia-related neurological disorder such as Alzheimer's disease (AD) or Vascular dementia (VaD).

29. The method of claim 27, wherein step d) comprises identifying that the subject has complied or adhered with the prescribed treatment regimen if the level of clusterin in c) compared to b) is increased and the subject has Alzheimer's disease or is at increased risk of developing Alzheimer's disease.

30. The method of claim 27, wherein step d) comprises identifying that the subject has complied or adhered with the prescribed treatment regimen if the level of clusterin in c) compared to b) is decreased and the subject has Vascular dementia or is at increased risk of developing Vascular dementia.

31. The method of claim 23, wherein the treatment regimen comprises at least one anti-dementia compound.

32. The method of claim 31, wherein the anti-dementia compound is:
a) a cholinesterase inhibitor, optionally wherein the cholinesterase inhibitor is selected from the group consisting of donepezil, rivastigmine, galantamine, tacrine, or salts thereof, and/or b) an NMDA antagonist, optionally wherein the NMDA antagonist is memantine.

33. The method of claim 22, wherein the level of at least two biomarkers selected from the group consisting of clusterin, alpha-synuclein and amyloid precursor protein is determined and compared.

34. The method of claim 22, wherein the level of biomarker is determined at the protein level, optionally using a process selected from the group consisting of immunoblotting, lateral flow assay, ELISA assay, protein microarray and mass spectrometry.

35. The method of claim 22, wherein the subject is a human.

36. Use of one or more biomarkers selected from the group consisting of clusterin, alpha-synuclein and amyloid precursor protein (APP) as a whole blood biomarker for assessing cognition in a subject having or at risk of having dementia.

37. The use according to claim 36, wherein the cause of the dementia is a dementia-related neurological disorder such as Alzheimer's disease (AD), Vascular dementia (VaD) or Dementia with Lewy bodies (DLB).

38. An in vitro method for monitoring changes in cognition in a subject having or at risk of having dementia, the method comprising the steps of:
i) performing the following steps:
a) providing a whole blood sample from the subject;
b) determining the level of one or more biomarker in the whole blood sample, wherein the one or more biomarker is selected from the group consisting of clusterin, alpha synuclein (AS) and amyloid precursor protein (APP);
c) comparing the level of the one or more biomarker with the level of the same biomarker in a control sample or with a pre-determined reference level for the same biomarker to identify an increase or decrease in a level of the one or more biomarker in the sample of the subject;
ii) repeating i) for the same subject after a time interval; and
iii) comparing the biomarker levels identified in i) with the biomarker levels identified in ii), and identifying a reduction in cognitive score if the comparison in step iii) indicates that the subject has one or more of the following: a change in the level of clusterin over the time interval; an increase in the level of alpha-synuclein over the time interval; or a decrease in the level of amyloid precursor protein over the time interval.

39. The method of claim 38, wherein the level of at least two biomarkers selected from the group consisting of clusterin, alpha-synuclein and amyloid precursor protein are determined and compared over the time interval.

40. The method of claim 38, wherein the cause of the dementia is a dementia-related neurological disorder such as Alzheimer's disease (AD), Vascular dementia (VaD) or dementia with Lewy bodies.

41. The method of claim 38, wherein the level of biomarker is determined at the protein level, optionally using a process selected from the group consisting of immunoblotting, lateral flow assay, ELISA assay, protein microarray and mass spectrometry.

42. The method of claim 38, wherein the control sample is obtained from a non-demented control subject.

43. The method of claim 38, wherein the pre-determined reference level is the average level of the biomarker in a non-demented control subject.

44. The method of claim 38, wherein the subject is a human.
